SEQUENCE LISTING (84 sequences)

SEQ ID NO: 1 - DNA, 19 bp, Artificial Sequence
  Forward primer
aggcggctctcttctctca

SEQ ID NO: 2 - DNA, 25 bp, Artificial Sequence
  Reverse primer
ctcctcctctgttgaaatggattct

SEQ ID NO: 3 - DNA, 39 bp, Artificial Sequence
  Forward primer
ttgttctaagtaccatatataacagaatgtttaaatatc

SEQ ID NO: 4 - DNA, 25 bp, Artificial Sequence
  Reverse primer
cccaggtctcctacttgttgttact

SEQ ID NO: 5 - DNA, 30 bp, Artificial Sequence
  Forward primer
tgactggtgtttacaaaagatattgtgaca

SEQ ID NO: 6 - DNA, 33 bp, Artificial Sequence
  Reverse primer
tactcatgcattttgaatactctcattaagtaa

SEQ ID NO: 7 - DNA, 19 bp, Artificial Sequence
  Forward primer
tgttggtggctgtgactgt

SEQ ID NO: 8 - DNA, 23 bp, Artificial Sequence
  Reverse primer
gggtctgaatccaggtagtctga

SEQ ID NO: 9 - DNA, 23 bp, Artificial Sequence
  Forward primer
ggaatggttttgactcggactca

SEQ ID NO: 10 - DNA, 22 bp, Artificial Sequence
  Reverse primer
tggcacatgtttgtcactgtct

SEQ ID NO: 11 - DNA, 17 bp, Artificial Sequence
  Forward primer
gcccacactccctcagc

SEQ ID NO: 12 - DNA, 17 bp, Artificial Sequence
  Reverse primer
gcgaagcccagctagga

SEQ ID NO: 13 - DNA, 24 bp, Artificial Sequence
  Forward primer
ccacaaataggcctctctcctctt

SEQ ID NO: 14 - DNA, 17 bp, Artificial Sequence
  Reverse primer
cctgggtgtggcttcgt

SEQ ID NO: 15 - DNA, 25 bp, Artificial Sequence
  Forward primer
ggtttaaagctcagtttcagcgttt

SEQ ID NO: 16 - DNA, 24 bp, Artificial Sequence
  Reverse primer
aggaagctacactgagagttgtga

SEQ ID NO: 17 - DNA, 33 bp, Artificial Sequence
  Forward primer
gctgtaagtacaaagttatttgatttgtagtgt

SEQ ID NO: 18 - DNA, 25 bp, Artificial Sequence
  Reverse primer
aatgaatagtagtgctgcacatcca

SEQ ID NO: 19 - DNA, 25 bp, Artificial Sequence
  Forward primer
acagtgatagcaggattggtagtga

SEQ ID NO: 20 - DNA, 18 bp, Artificial Sequence
  Reverse primer
gcccacgccagtagagaa

SEQ ID NO: 21 - DNA, 28 bp, Artificial Sequence
  Forward primer
agcagggccagataatgtttatgataat

SEQ ID NO: 22 - DNA, 28 bp, Artificial Sequence
  Reverse primer
ccattcaataaatactgagccaggagta

SEQ ID NO: 23 - DNA, 18 bp, Artificial Sequence
  Forward primer
tcctgccctgaggtctga

SEQ ID NO: 24 - DNA, 24 bp, Artificial Sequence
  Reverse primer
cttatcctcccagctcttcatctg

SEQ ID NO: 25 - DNA, 31 bp, Artificial Sequence
  Forward primer
ccatttgatggtcatgagactaatgttatct

SEQ ID NO: 26 - DNA, 28 bp, Artificial Sequence
  Reverse primer
agaatgtgaccataactatcgactgagt

SEQ ID NO: 27 - DNA, 28 bp, Artificial Sequence
  Forward primer
aatgcttatctttctggtcatgagtctt

SEQ ID NO: 28 - DNA, 40 bp, Artificial Sequence
  Reverse primer
ggatatatttatacatgataaaaacagtagtggttcctaa

SEQ ID NO: 29 - DNA, 26 bp, Artificial Sequence
  Forward primer
ccttgttgcttttgatacactctcat

SEQ ID NO: 30 - DNA, 25 bp, Artificial Sequence
  Reverse primer
cttgcccaagatcacatgtctagta

SEQ ID NO: 31 - DNA, 25 bp, Artificial Sequence
  Forward primer
agaagtggacataaggcctttagtg

SEQ ID NO: 32 - DNA, 21 bp, Artificial Sequence
  Reverse primer
ttcccactgcacaagcataca

SEQ ID NO: 33 - DNA, 42 bp, Artificial Sequence
  Forward primer
ctttttttttctaaggtaaaattagtaacacatttatttggg

SEQ ID NO: 34 - DNA, 28 bp, Artificial Sequence
  Reverse primer
tcagaaatagcatatgtttttggaggct

SEQ ID NO: 35 - DNA, 27 bp, Artificial Sequence
  Forward primer
caaaagtgacacttaccccaattacag

SEQ ID NO: 36 - DNA, 20 bp, Artificial Sequence
  Reverse primer
gctgttcgtgtcgtctcctt

SEQ ID NO: 37 - DNA, 25 bp, Artificial Sequence
  Forward primer
gacagggctttatgtattagccaca

SEQ ID NO: 38 - DNA, 18 bp, Artificial Sequence
  Reverse primer
gcacacgccctgagaaca

SEQ ID NO: 39 - DNA, 18 bp, Artificial Sequence
  Forward primer
gcgctgcggaagatcatc

SEQ ID NO: 40 - DNA, 22 bp, Artificial Sequence
  Reverse primer
gcttgagggtctgaatcttgct

SEQ ID NO: 41 - DNA, 28 bp, Artificial Sequence
  Forward primer
acatgggtgatgaactagaatttgaagt

SEQ ID NO: 42 - DNA, 27 bp, Artificial Sequence
  Reverse primer
tgtgatatttagttttccccgaatgca

SEQ ID NO: 43 - DNA, 26 bp, Artificial Sequence
  Forward primer
agattggtaagctagcaatgaatgct

SEQ ID NO: 44 - DNA, 25 bp, Artificial Sequence
  Reverse primer
cgtctaagctcacacttgaaaggta

SEQ ID NO: 45 - DNA, 22 bp, Artificial Sequence
  Forward primer
acttactacacctggccatgga

SEQ ID NO: 46 - DNA, 29 bp, Artificial Sequence
  Reverse primer
aacattttaagcactgcagtaaatttggt

SEQ ID NO: 47 - DNA, 22 bp, Artificial Sequence
  Forward primer
tgctggacactttcacttggaa

SEQ ID NO: 48 - DNA, 25 bp, Artificial Sequence
  Reverse primer
gagaggacttcctagggaacttagg

SEQ ID NO: 49 - DNA, 22 bp, Artificial Sequence
  Forward primer
agtttgggaagccctcctctaa

SEQ ID NO: 50 - DNA, 26 bp, Artificial Sequence
  Reverse primer
tcaccttctgatgaacacttttctgt

SEQ ID NO: 51 - DNA, 20 bp, Artificial Sequence
  Forward primer
gcctcagtgggaacttctgt

SEQ ID NO: 52 - DNA, 23 bp, Artificial Sequence
  Reverse primer
catgcctttattcccagcttctc

SEQ ID NO: 53 - DNA, 22 bp, Artificial Sequence
  Forward primer
aggaaaccttcaggagcaactc

SEQ ID NO: 54 - DNA, 22 bp, Artificial Sequence
  Reverse primer
ccttgagcagagacgtctttca

SEQ ID NO: 55 - DNA, 15 bp, Artificial Sequence
  Forward primer
gcccctggtgcgaca

SEQ ID NO: 56 - DNA, 20 bp, Artificial Sequence
  Reverse primer
ccaagtgcctcaaagttgca

SEQ ID NO: 57 - DNA, 16 bp, Artificial Sequence
  Probe
ttgctgctgctctttc

SEQ ID NO: 58 - DNA, 16 bp, Artificial Sequence
  Probe
ttgtctgctgaatttt

SEQ ID NO: 59 - DNA, 16 bp, Artificial Sequence
  Probe
atgccacgtacatttt

SEQ ID NO: 60 - DNA, 15 bp, Artificial Sequence
  Probe
cccacagggctcacc

SEQ ID NO: 61 - DNA, 16 bp, Artificial Sequence
  Probe
ccctcactggttctcc

SEQ ID NO: 62 - DNA, 16 bp, Artificial Sequence
  Probe
cccacacgctcctctc

SEQ ID NO: 63 - DNA, 16 bp, Artificial Sequence
  Probe
tccctcctgatgcccc

SEQ ID NO: 64 - DNA, 15 bp, Artificial Sequence
  Probe
caggtgtccaaattc

SEQ ID NO: 65 - DNA, 16 bp, Artificial Sequence
  Probe
ctcctgctaaaatttc

SEQ ID NO: 66 - DNA, 16 bp, Artificial Sequence
  Probe
atgctgggcggcactt

SEQ ID NO: 67 - DNA, 15 bp, Artificial Sequence
  Probe
ctgccacggaagtat

SEQ ID NO: 68 - DNA, 16 bp, Artificial Sequence
  Probe
ccagcctgaggaccag

SEQ ID NO: 69 - DNA, 16 bp, Artificial Sequence
  Probe
aaggccagaaaatcac

SEQ ID NO: 70 - DNA, 16 bp, Artificial Sequence
  Probe
taggcatcccaagctc

SEQ ID NO: 71 - DNA, 16 bp, Artificial Sequence
  Probe
caatcagcccaaattt

SEQ ID NO: 72 - DNA, 15 bp, Artificial Sequence
  Probe
cccagcctgaattag

SEQ ID NO: 73 - DNA, 16 bp, Artificial Sequence
  Probe
atggcaggcactaaac

SEQ ID NO: 74 - DNA, 16 bp, Artificial Sequence
  Probe
cagcccgtcattcgca

SEQ ID NO: 75 - DNA, 16 bp, Artificial Sequence
  Probe
cccctcctcccttctc

SEQ ID NO: 76 - DNA, 15 bp, Artificial Sequence
  Probe
ccctcggacaagctg

SEQ ID NO: 77 - DNA, 17 bp, Artificial Sequence
  Probe
atgctgctggagtattc

SEQ ID NO: 78 - DNA, 14 bp, Artificial Sequence
  Probe
ctcaccagcttccc

SEQ ID NO: 79 - DNA, 19 bp, Artificial Sequence
  Probe
tcttcttgtgtcaactttg

SEQ ID NO: 80 - DNA, 21 bp, Artificial Sequence
  Probe
cctccgtatactttctatccc

SEQ ID NO: 81 - DNA, 21 bp, Artificial Sequence
  Probe
ctgagcacatttatacagatg

SEQ ID NO: 82 - DNA, 14 bp, Artificial Sequence
  Probe
cctgcccgctctgc

SEQ ID NO: 83 - DNA, 16 bp, Artificial Sequence
  Probe
tcacactggagaggcc

SEQ ID NO: 84 - DNA, 18 bp, Artificial Sequence
  Probe
ttctgtggcagccagctg

(19) United States
(12) Patent Application Publication
(10) Pub. No.: US 2014/0242580 A1
(43) Pub. Date: Aug. 28, 2014

(54) METHOD FOR PREDICTING RESPONSE OR PROGNOSIS OF LUNG ADENOCARCINOMA WITH EGFR-ACTIVATING MUTATIONS

(71) Applicants: Sung-liang Yu, Taipei City (TW); Pan-chyr Yang, Taipei City (TW); Shinsheng Yuan, Taoyuan County (TW); Gee-chen Chang, Taichung City (TW); Hsuan-yu Chen, Taipei City (TW); Ker-chau Li, Taipei City (TW)

(72) Inventors: Sung-liang Yu, Taipei City (TW); Pan-chyr Yang, Taipei City (TW); Shinsheng Yuan, Taoyuan County (TW); Gee-chen Chang, Taichung City (TW); Hsuan-yu Chen, Taipei City (TW); Ker-chau Li, Taipei City (TW)

(73) Assignee: NATIONAL TAIWAN UNIVERSITY, Taipei (TW)

(21) Appl. No.: 14/131,182

(22) PCT Filed: Jul. 5, 2012
(86) PCT No.: PCT/IB2012/001595

§ 371 Date: Feb. 11, 2014

Related U.S. Application Data
(60) Provisional application No. 61/504,512, filed on Jul. 5, 2011.

(51) Int. Cl. C12Q 1/68

(52) U.S. Cl. CPC C12Q 1/68; C12Q 2539/10
USPC 435/6.11

(57) ABSTRACT

The invention provides a method for predicting the response of an EGFR-activating mutant subject suffering from a lung adenocarcinoma and receiving treatment with epidermal growth factor receptor tyrosine kinase inhibitor (EGFR-TKI) and a method for predicting prognosis in an EGFR-activating mutant subject suffering from a lung adenocarcinoma and receiving treatment with EGFR-TKI. In the methods of the invention, clustered genomic alterations in specific chromosomes (in particular chromosomes 5p, 7p, 8q or 14q) are determined as a tool for predicting the response or prognosis.

### FIELD OF THE INVENTION

The invention provides a method for predicting the response of an EGFR-activating mutant subject suffering from a lung adenocarcinoma and receiving treatment with epidermal growth factor receptor tyrosine kinase inhibitor (EGFR-TKI) and a method for predicting prognosis in an EGFR-activating mutant subject suffering from a lung adenocarcinoma and receiving treatment with EGFR-TKI. Particularly, clustered genomic alterations in specific chromosomes are determined as a tool for predicting the response or prognosis in the methods.

### BACKGROUND OF THE INVENTION

Lung adenocarcinoma is the predominant type of lung cancer and is the most common cause of cancer deaths worldwide. Among all histological types of lung cancer, adenocarcinoma is the most common and has the greatest heterogeneity.

Treatment of lung adenocarcinoma (such as Non-small-cell lung cancer; NSCLC) has been relatively poor. Chemotherapy, the mainstay treatment of advanced cancers, is only marginally effective, with the exception of localized cancers. While surgery is the most potentially curative therapeutic option for lung adenocarcinoma, it is not always possible depending on the stage of the cancer. Recent approaches for developing anti-cancer drugs to treat the lung adenocarcinoma patients focus on reducing or eliminating the cancer cells' ability to grow and divide. These anti-cancer drugs are used to disrupt the signals which tell the cells to grow or die. Normally, cell growth is tightly controlled by the signals that the cells receive. In cancer, however, this signaling goes wrong and the cells continue to grow and divide in an uncontrollable fashion, thereby forming a tumor. One of these signaling pathways begins when a protein, called epidermal growth factor (EGF), binds to a receptor that is found on the surface of many cells.

EGFR is a member of the type 1 tyrosine kinase family of growth factor receptors, which play a critical role in cellular growth, differentiation and survival. Activation of these receptors typically occurs via specific ligand binding, resulting in hetero- or homodimerization between receptor family members, with subsequent autophosphorylation of the tyrosine kinase domain. Mutations of EGFR are present in a subpopulation of NSCLC patients. EGFR mutation rate is higher in East Asian patients (19-26%) than in those of European or US descent (8-17%). EGFR-mutation mediated phosphorylation can activate downstream anti-apoptotic signal transduction via Akt pathway or proliferative signals via MAPK/ERK pathway. Strikingly, patients with NSCLC harboring these genetic alterations revealed a remarkable response to EGFR-Tyrosine Kinase Inhibitors (TKIs) and the treatment efficacy was confirmed in clinical trials (Maemondo M, et al: Gefitinib or chemotherapy for non-small-cell lung cancer with mutated EGFR. N Engl J Med 362:2380-8, 2010; Lynch T J, et al: Activating mutations in the epidermal growth factor receptor underlying responsiveness of non-small-cell lung cancer to gefitinib. N Engl J Med 350:2129-39, 2004; Paez J G, et al: EGFR mutations in lung cancer: correlation with clinical response to gefitinib therapy. Science 304:1497-500, 2004; Mitsudomi T, et al: Gefitinib versus cisplatin plus docetaxel in patients with non-small-cell lung cancer harbouring mutations of the epidermal growth factor receptor (WJTOG3405): an open label, randomised phase 3 trial. Lancet Oncol 11:121-8, 2010; Mok T S, et al: Gefitinib or Carboplatin-Paclitaxel in Pulmonary Adenocarcinoma. N Engl J Med 361:947-957, 2009). High response rate may be due to EGFR mutations within critical residues of the catalytic domain, causing physical structure alteration in drug binding (Yun C H, et al: Structures of lung cancer-derived EGFR mutants and inhibitor complexes: mechanism of activation and insights into differential inhibitor sensitivity. Cancer Cell 11:217-27, 2007). U.S. Pat. No. 7,932,026 teaches mutations in EGFR and methods of detecting such mutations as well as prognostic methods for identifying a tumor that is susceptible to anticancer therapy such as chemotherapy and/or kinase inhibitor treatment.

Although several studies have established that the EGFR-TKIs are in general more effective for patients with EGFR-activating mutations than EGFR wild-type, the responses are quite heterogeneous even among the EGFR mutant patients (Mok T S, et al: Gefitinib or Carboplatin-Paclitaxel in Pulmonary Adenocarcinoma. N Engl J Med 361:947-957, 2009). The IPASS study reported that only 71% of patients with EGFR activating mutation responded well to EFGR-TKIs (Mok T S, et al: Gefitinib or Carboplatin-Paclitaxel in Pulmonary Adenocarcinoma. N Engl J Med 361:947-957, 2009). To identify non-responsive patients, U.S. Pat. No. 7,858,389 provides methods using mass spectral data analysis and a classification algorithm provide an ability to determine whether a non-small-cell lung cancer (NSCLC) patient is likely to benefit from a monoclonal antibody drug targeting an epidermal growth factor receptor pathway. U.S. Pat. No. 7,906,342 provides methods using mass spectral data analysis and a classification algorithm provide an ability to determine whether a non-small-cell lung cancer patient, head and neck squamous cell carcinoma or colorectal cancer patient has likely developed a non-responsiveness to treatment with a drug targeting an epidermal growth factor receptor pathway. However, these prior art references use mass spectrum obtained from a blood sample as the tool for identification and the effects are not satisfactory.

Since the molecular basis of the response heterogeneity is still unknown and no biomarker is available for response prediction, there remains a need for a technique for predicting responsiveness of a lung adenocarcinoma subject receiving EGFR treatment.

### SUMMARY OF THE INVENTION

The invention relates to a method for predicting the response of an EGFR-activating mutant subject suffering from a lung adenocarcinoma and receiving treatment with epidermal growth factor receptor tyrosine kinase inhibitor (EGFR-TKI), comprising a) providing a sample comprising genomic DNA from said EGFR-activating mutant subject; and b) analyzing said genomic DNA to determine copy number alterations (CNAs) of genes in chromosome 5p, 7p, 8q or 14q of the sample, wherein changes of CNAs in the sample of a) relative to a sample comprising genomic DNA of a EGER wild-type indicate that the EGFR-activating mutant subject has less favorable response to treatment with the EGFR-TKI.

The invention also relates to a method of predicting prognosis in an EGFR-activating mutant subject suffering from a lung adenocarcinoma and receiving treatment with EGFR-TKI, comprising a) providing a sample comprising genomic DNA from said EGFR-activating mutant subject; and b) analyzing said genomic DNA to determine copy number alterations (CNAs) of genes in chromosome 5p, 8q or 14q of the sample, wherein the subject is determined to have poorer prognosis when the CNAs in the sample of a) is changed relative to the CNAs of genes in a sample comprising genomic DNA of an EGFR wild-type.

The invention further relates to a diagnostic kit for determining the response of a EGFR-activating mutant subject suffering from lung adenocarcinoma and receiving treatment with EGFR-TKI, or determining prognosis in an EGFR-activating mutant subject suffering from a lung adenocarcinoma and receiving treatment with EGFR-TKI, comprising one or more probes to the genes in chromosome 5p, 8q or 14q of the sample comprising genomic DNA from said EGFR-activating mutant subject.

### BRIEF DESCRIPTION OF THE DRAWING

FIG. 1. Sites of differential CNA found in EGFR-activating mutation status comparisons. The sites of probe-blocks displaying the differential CNA in three comparisons, the EGFR-activating mutant group versus the wild-type group, the L858R mutant group versus the EGFR wild-type group and the exon-19 in-frame deletion group versus EGFR wild-type group are shown on the right side of each chromosome ideogram. A zoom-in version of chromosome 7p is given on the right, along with the locations of some notable genes.

FIG. 2. Representative CNA profiles on chromosome 7p for the EGFR-activating mutation group and the EGFR wild-type group of lung adenocarcinoma.

FIG. 3. The Kaplan-Meier curves for both overall survival and progression-free survival analysis are provided. The clinical variables considered are EGFR mutation status, stage, age, gender and smoking status.

FIG. 4. Survival prediction by DNA copy numbers of six genes from chromosome 7p. (A) Patients are listed in an ascending order from left to right based on the CNA-risk scores. The survival time of each patient is plotted in the top panel. The bottom panel shows the copy numbers of six genes in a heat map. Pale blue dotted line represents the median of CNA-risk score dividing patients into low risk and high risk signature groups. (B) The Kaplan-Meier curves for both overall survival and progression-free survival analyses on EGFR-activating mutation patients are shown. The high and low risk groups are divided evenly based on the CNA-risk scores. (C) Same analysis as (B), applied to the EGFR wild-type group of patients.

FIG. 5. (A) Box plot for CNA-risk score distribution. Significant difference between favorable responders (partial response, 11 cases) and less favorable responders (progressive disease or stable disease, 12 cases) is shown. Two-sided t-test p value is given. (B) EGFR-TKI treatment responsiveness is associated with copy number increase in multiple genes on chromosome 7p. The Fisher exact test p value is given.

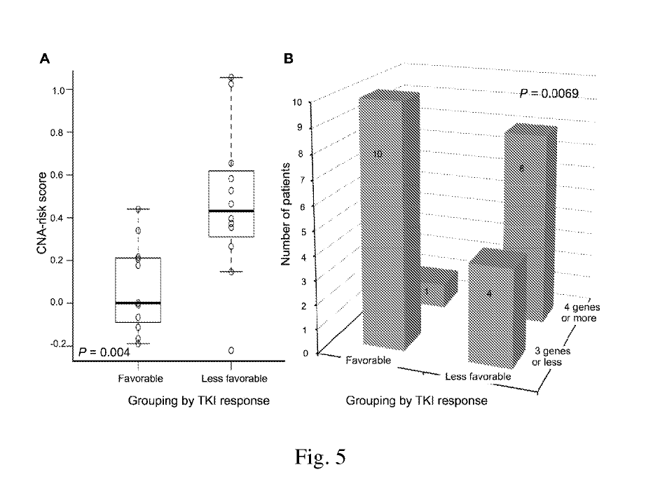

### DETAILED DESCRIPTION OF THE INVENTION

The invention identifies chromosome regions with differential copy number alterations (CNAs) between the EGFR-activating mutant and EGFR wild-type tumors and found the aberration sites to cluster highly on chromosome 5p, 7p, 8q or 14q. A cluster of chromosome genes predicts the overall and the progression-free survivals for EGFR-activating mutant patients, but not wild-type. Importantly, presence of genes with changed CNA in this cluster correlates with less favorable response to EGFR-TKIs in EGFR-activating mutant patients.

Unless otherwise defined, scientific and technical terms used in connection with the present invention shall have the meanings that are commonly understood by those of ordinary skill in the art. Further, the singular form “a,” “an,” and “the” include plural references unless the context clearly dictates otherwise.

As used herein, a “subject” refers to a vertebrate mammal, including, but not limited to, human, mouse, rat, dog, cat, horse, cow, pig, sheep, goat, or non-human primate. In some embodiments, the subject is a human. The terms “subject,” “patient” and “individual” are used interchangeably.

As used herein, a “genome” designates or denotes the complete, single-copy set of genetic instructions for an organism as coded into the DNA of the organism. A genome may be multi-chromosomal so that the DNA is cellularly distributed among a plurality of individual chromosomes. For example, in human there are 22 pairs of chromosomes plus a gender associated XX or XY pair.

As used herein, the “EGFR mutant” or “EGFR mutations” means an amino acid or nucleic acid sequence that differs from wild-type EGFR protein or nucleic acid respectively found on one allele (heterozygous) or both alleles (homozygous) and may be somatic or germ line. In an embodiment, said mutation is an amino acid or nucleic acid substitution, deletion or insertion.

As used herein, the “chromosome” refers to the heredity-bearing gene carrier of a living cell which is derived from chromatin and which comprises DNA and protein components (especially histones). The conventional and internationally recognized individual human genome chromosome numbering system is employed herein. The size of an individual chromosome can vary from one type to another with a given multi-chromosomal genome and from one genome to another.

As used herein, the “chromosomal region” is a portion of a chromosome. The actual physical size or extent of any individual chromosomal region can vary greatly. The term “region” is not necessarily definitive of a particular one or more genes because a region need not take into specific account the particular coding segments (exons) of an individual gene.

As used herein, the “copy number” of a nucleic acid refers to the number of discrete instances of that nucleic acid in a given sample.

As used herein, the “copy number alteration” refers to a variation in the number of copies of a gene or genetic region that is present in the genome of a cell. A normal diploid cell will typically have two copies of each chromosome and the genes contained therein. Copy number alterations may increase the number of copies, or decrease the number of copies.

As used herein, “copy number profile” means a collection of data representing the number of copies of genomic DNA at a plurality of genomic loci for a given sample. For instance, for three genomic loci of interest, a copy number profile represents the number of copies of DNA for the three genomic loci. In this context, “genomic locus” means a location within the genome of a cell and usually encompasses a stretch of genomic DNA between two points in the genome of a cell. This stretch of genomic DNA consists of a nucleotide sequence.

As used herein, the “prognosis” is meant response and/or benefit and/or survival.

In one aspect, the invention provides a method for predicting the response of an EGFR-activating mutant subject suffering from a lung adenocarcinoma and receiving treatment with epidermal growth factor receptor tyrosine kinase inhibitor (EGFR-TKI), comprising a) providing a sample comprising genomic DNA from said EGFR-activating mutant subject; and b) analyzing said genomic DNA to determine copy number alterations (CNAs) of genes in chromosome 5p, 7p, 8q or 14q of the sample, wherein changes of CNAs in the sample of a) relative to a sample comprising genomic DNA of an EGFR wild-type indicates that the EGFR-activating mutant subject has less favorable response to treatment with the EGFR-TKI.

In another aspect, the invention provides a method of predicting prognosis in a EGFR-activating mutant subject suffering from a lung adenocarcinoma and receiving treatment with EGFR-TKI, comprising a) providing a sample comprising genomic DNA from said EGFR-activating mutant subject; and b) analyzing said genomic DNA to determine copy number alterations (CNAs) of genes in chromosome 5p, 7p, 8q or 14q of the sample, wherein the subject is determined to have poorer prognosis when the CNAs in the sample of a) change relative to the CNAs of genes in a sample comprising genomic DNA of an EGFR wild-type.

In a further aspect, the invention provides a diagnostic kit for determining the response of an EGFR-activating mutant subject suffering from lung adenocarcinoma and receiving treatment with EGFR-TKI, or determining prognosis in an EGFR-activating mutant subject suffering from a lung adenocarcinoma and receiving treatment with EGFR-TKI, comprising one or more probes to the genes in chromosome 5p, 7p, 8q or 14q of the sample comprising genomic DNA from said EGFR-activating mutant subject. The kits can additionally include instructional materials describing when and how to use the kit contents. The kits can also include one or more of the following: various labels or labeling agents to facilitate the detection of the probes, reagents for the hybridization including buffers, a metaphase spread, bovine serum albumin (BSA) and other blocking agents, sampling devices including fine needles, swabs, aspirators and the like, positive and negative hybridization controls and so forth.

According to the invention, EGFR tyrosine kinase inhibitors bind the ATP binding pocket of the EGFR receptor and prevent ATP from binding. As a result, binding of the inhibitor results in the suppression of EGFR mediated intracellular signaling. EGFR tyrosine kinase inhibitors include both reversible and irreversible inhibitors. Most reversible inhibitors are based on quinazolines and include, but are not limited to, gefitinib (Iressa; N-(3-Chloro-4-fluoro-phenyl)-7-methoxy-6-(3-morpholin-4-ylpropoxy)quinazo-lin-4-amine), erlotinib (Tarceva; N-(3-ethynylphenyl)-6,7-bis(2-methoxyethoxy)quinazolin-4-amine) and lapatinib (Tykerb, GW572016; N-[3-chloro-4-[(3-fluorophenyl)methoxy]phenyl]-6-[5-[(2-methylsulfonyleth-ylamino)methyl]-2-furyl]quinazolin-4-amine) Irreversible inhibitors permanently modify the tyrosine kinase domain of EGFR, thereby suppressing EGFR signaling. Irreversible inhibitors include, but are not limited to, CI-1033, EKB-569 and HKI-272 (See e.g., Zhang et al., 2007, JCI 117: 2051-2058). The binding of an EGFR-TKI to EGFR leads to the induction of apoptosis of the cell expressing the EGFR, thereby providing a method for cancer treatment. It should be appreciated that the terms EGFR tyrosine kinase inhibitor and EGFR kinase inhibitor are used interchangeably herein.

According to one embodiment of the invention, the lung adenocarcinoma is NSCLC.

According to the invention, the copy number alterations (CNAs) of genes change in chromosome 5p, 7p, 8q or 14q of the sample comprising genomic DNA from an EGFR-activating mutant subject. Preferably, the CNAs change in the chromosome 7p. More preferably, the CNAs change in the chromosome 7p11.2, 7p14.1, 7p15.2, 7p15.3, 8q11.21 or 8q11.23. More preferably, the CNAs change in one or more of the following representative genes, EGFR, LANCL2, VSTM2A, VOPP1, SEC61G, SEPT14 and HPVC1 located at the chromosome 7p11.2, GLI3 and C7orf10 located at the chromosome 7p14.1, NFE2L3, MIR148A and OSBPL3 located at the chromosome 7p15.2, NPY located at the chromosome 7p15.3, SDK1 located at the chromosome 7p22.2, ANK1 located at the chromosome 8p11.21 and ADAM3A located at the chromosome 8p11.23. Most preferably, the CNAs change in one or more of the six representative genes, GLI3, NFE2L3, SDK1, EGFR, VOPP1 and LANCL2 located at the chromosome 7p14.1, 7p15.2, 7p22.2, 7p11.2, 7p11.2 and 7p11.2, respectively.

In one embodiment of the invention, the changes of CNAs are DNA gain in chromosome 5p, 7p or 14q and DNA loss in chromosome 8q.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

Nucleic acid hybridization assays for the detection of target region sequences, for quantifying copy number, for sequencing, and the like, can be performed in an array-based format (such as comparative genomic hybridization (Cgh) using nucleic acid arrays). Arrays are a multiplicity of different “probe” or “target” nucleic acids (or other compounds) hybridized with a sample nucleic acid. In an array format a large number of different hybridization reactions can be run in parallel. This provides rapid, essentially simultaneous, evaluation of a large number of loci.

The nucleic acid probes are fixed to a solid surface in an array. These probes comprise portions of the target regions of the invention, optionally in combination with probes from other portions of the genome. Probes can be obtained from any convenient source, including MACs, YACs, BACs, PACs, cosmids, plasmids, inter-Alu PCR products of genomic clones, restriction digests of genomic clones, CDNA clones, amplification products, and the like. The arrays can be hybridized with a single population of sample nucleic acid or can be used with two differentially labeled collections, for example a test sample and a reference sample.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g. nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, cermets or the like. In addition, substances that form gels can be used. Such materials include proteins, lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins such as casein or BSA or mixtures of macromolecules can be employed to avoid non-specific binding, simplify covalent conjugation, enhance signal detection or the like. If the probe is to be covalently bound, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. For example, methods for immobilizing nucleic acids by introduction of various functional groups to the molecules are known. Covalent attachment of the target nucleic acids to glass or synthetic fused silica can be accomplished according to a number of known techniques and commercially available reagents. For instance, materials for preparation of silanized glass with a number of functional groups are commercially available or can be prepared using standard techniques. Quartz cover slips, which have at least 10-fold lower autofluorescence than glass, can also be silanized.

Alternatively, probes can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling. Hybridization to nucleic acids attached to beads is accomplished by suspending them in the hybridization mix, and then depositing them on a substrate for analysis after washing, or analyzing by flow cytometry.

Comparative genomic hybridization (CGH) can detect and map DNA sequence copy number variation throughout the entire genome in a single experiment. In one variation of CGH, the genome is provided as a cytogenetic map through the use of metaphase chromosomes. Alternatively hybridization probes are arrays of genomic sequences containing the target region sequences of the invention, optionally also including other genomic probes. Relative copy number can also be measured by hybridization of fluorescently labeled test and reference nucleic acids in both metaphase chromosome-based and array-based CGH.

In metaphase chromosome-based CGH total genomic DNA is isolated from a sample of a subject, labeled with different fluorochromes, and hybridized to normal metaphase chromosomes. Cot-1 DNA is used to suppress hybridization of repetitive sequences. The resulting ratio of the fluorescence intensities of the two fluorochromes at a location on a chromosome is approximately proportional to the ratio of the copy numbers of the corresponding DNA sequences in the test and reference genomes. Thus, CGH provides genome-wide copy number analysis referenced to the cytogenetic map provided by the metaphase chromosomes. However, the use of metaphase chromosome CGH limits the resolution to 10-20 megabases (Mb), prohibits resolution of closely spaced aberrations, and only allows linkage of CGH results to genomic information and resources with cytogenetic accuracy.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal, for example antibody-antigen or complementary nucleic acid binding. The label may also allow indirect detection of the hybridization complex. For example, where the label is a hapten or antigen, the sample can be detected by using antibodies. In these systems, a signal is generated by attaching fluorescent or radioactive label or enzymatic molecule to the antibodies. The sensitivity of the hybridization assays can be enhanced through use of a target nucleic acid or signal amplification system that multiplies the target nucleic acid or signal being detected. Alternatively, sequences can be generally amplified using nonspecific PCR primers and the amplified target region later probed for a specific sequence indicative of a mutation.

Various other technologies may also be used for determining copy number. In some embodiments, the method involves amplifications of a test locus with unknown copy number and a reference locus with known copy number using real-time PCR. Progress in the PCR reactions is monitored using fluorigenic probes and a real-time fluorescence detection system. For each reaction, the number of cycles is measured at which a defined threshold fluorescence emission is reached. Using standard curves, the copy number of the test DNA relative to a common standard DNA is determined for each locus. From the ratio of the relative copy numbers, the genomic copy number of the test locus is determined (see Wilke et al. (2000) Hum Mutat 16:431-436).

The results provided in the invention shed light on why among patients with EGFR mutation, responses to the EGFR TKI-targeted therapy are heterogeneous. This may lead to a better patient management for EGFR-mutant patients. The invention provides data to highlight chromosome 5p, 7p, 8q or 14q as the main chromosome arm enriched in notable sites of DNA copy number alterations for lung adenocarcinoma, so it is an effective predictor for both overall survival and progression-free survival of EGFR mutant patients. In this connection, chromosome 7p is the preferred embodiment. Furthermore, the invention shows that six qPCR-validated genes from chromosome 7p yield a copy-number based risk score which is an effective predictor for both overall survival and progression-free survival of EGFR mutant patients, independent of cancer staging. Yet for the EGFR wild-type patients, the invention also shows that the same signature is uncorrelated with both the overall survival and progression-free survival. This sharp contrast strongly supports the useful notion of using EGFR-mutation status to define subtypes of adenocarcinoma.

To a clinician treating the lung cancer patients, differences in the patients' ethnic and pharmacogenomic backgrounds are important factors that may heavily influence the decision in the individualized therapy. The genetic alterations clustered in chromosome 5p, 7p, 8q or 14q region (in particular chromosome 7p) that the invention identified may play a crucial role and the risk score derived from these genetic alterations may determine whether the patient will have a favorable response to EGFR-TKI therapy. The invention provides clues to why patients with EGFR-activating mutation may still have heterogeneous response to EGFR-TKI targeted therapy. The finding may also be useful for clinician to make better prediction for the treatment response. The invention also suggests that in patients with EGFR driver gene mutation, the chromosome 5p, 7p, 8q or 14q region (in particular chromosome 7p) is more vulnerable to damage by carcinogen.

### EXAMPLE

While the invention has been described with reference to specific embodiments, it will be understood by those skilled in the art that various changes may be made and equivalents may be substituted for elements thereof to adapt to particular situations without departing from the scope of the invention. The following experimental examples are provided in order to demonstrate and further illustrate various aspects of certain embodiments of the present invention and are not to be construed as limiting the scope thereof. In the experimental disclosure which follows, the following materials and methods are used:

### Patients and Methods

The 138 cancer tissues for array CGH assay were obtained from National Taiwan University Hospital (NTUH) and Taichung Veterans General Hospital (TCVGH). The 114 cancer tissues for clinical outcome prediction by genomic real-time qPCR were obtained from TCVGH. There were no overlaps between these two groups of patients. After surgical operation, the dissected tissues from lung adenocarcinoma patients were stored in the liquid nitrogen immediately and anonymized. Following the standard protocol, the genomic DNA was extracted from cancer tissue of each sample with quality checked by agarose electrophoresis.

Tumor DNA from 25 EGFR mutant (exon-19 deletion and L858R) patients of TCVGH for EGFR-TKI was obtained for treatment response study. One squamous cell carcinoma patient and one patient with insufficient information were deleted. For the remaining 23 patients, three types of responses were evaluated by physicians according to the guideline of RECIST 1.0:1. partial response (PR), 2. progressive disease (PD), 3. stable disease (SD) (P. Therasse S G A, E. A. Eisenhauer: New Guidelines to Evaluate the Response to Treatment in Solid Tumors (RECIST Guidelines). Journal of the National Cancer Institute 92:205-216, 2000).

### Array Comparative Genomic Hybridization (CGH)

The whole genome NimbleGen CGH array (NimbleGen®; NimbleGen Systems Inc, Madison, Wis.) containing 385,806 probes with probe spacing of about 6,000 bp, was used for comparative genomic hybridization of DNA from cancer tissues against normal DNA extracted from the PBMC of one male and one female in a community cohort. Digital sonifier (Branson Model#450, Branson, Danbury, Conn.) was used for the DNA fragmentation. Labeling, hybridization and washing were processed according to the manufacturer's protocol. The array scanning and image generation were performed by the GenePix™ Reader (Personal 4000B, Axon Instruments, Molecular Devices, Sunnyvale, Calif.) and GenePix® Pro 6.0 software. Generation of log intensity ratio data with normalization was performed by NimbleScan™ version 2.4, SignalMap™ version 1.9 software, followed by applying cross-chip normalization. The original CNA dataset in dot pair format can be accessed at http://kiefer.stat2.sinica.edu.tw/cghdata/.

Genomic Real-Time Quantitative PCR (q-PCR)

qPCR has been established as a rapid and sensitive technique for accurate quantification of DNA in tissues. The fluorescence emitted by the reporter dye was detected on-line in real-time using the ABI prism 7900 sequence detection system (Applied Biosystem, Foster City, Calif.). The primers and probes of qPCR were designed based on 500 franking nucleotide sequences (250 upstream and 250 downstream nucleotides) of the probe location of array CGH. The sequences of primers and probes were given in Table 1.

TABLE 1
The probes and primers used for genomic real-time qPCR

| Gene | Forward primer | Reverse primer | Probe |
|---|---|---|---|
| EGFR | AGGCGGCTCTCT | CTCCTCCTCTGTTG | TTGCTGCTGCTCTTTC |
| | TCTCTCA | AAATGGATTCT | (SEQ ID NO: 57) |
| | (SEQ ID NO: 1) | (SEQ ID NO: 2) | |
| DGKB | TTGTTCTAAGTAC | CCCAGGTCTCCTA | TTGTCTGCTGAATTTT |
| | CATATATAACAGA | CTTGTTGTTACT | (SEQ ID NO: 58) |
| | ATGTTTAAATATC | (SEQ ID NO: 4) | |
| | CC | | |
| | (SEQ ID NO: 3) | | |
| MEOX2 | TGACTGGTGTTT | TACTCATGCATTTT | ATGCCACGTACATTTT |
| | ACAAAAGATATTG | GAATACTCTCATTA | (SEQ ID NO: 59) |
| | TGACA | AGTAA | |
| | (SEQ ID NO: 5) | (SEQ ID NO: 6) | |
| ERBB2 | TGTTGGTGGCTG | GGGTCTGAATCCA | CCCACAGGGCTCACC |
| | TGACTGT | GGTAGTCTGA | (SEQ ID NO: 60) |
| | (SEQ ID NO: 7) | (SEQ ID NO: 8) | |
| ACTN4 | GGAATGGTTTTG | TGGCACATGTTTGT | CCCTCACTGGTTCTCC |
| | ACTCGGACTCA | CACTGTCT | (SEQ ID NO: 61) |
| | (SEQ ID NO: 9) | (SEQ ID NO: 10) | |
| FAM102A | GCCCACACTCCC | GCGAAGCCCAGCT | CCCACACGCTCCTCTC |
| | TCAGC | AGGA | (SEQ ID NO: 62) |
| | (SEQ ID NO: 11) | (SEQ ID NO: 12) | |
| MEOX1 | CCACAAATAG | CCTGGGTGTGGCT | TCCCTCCTGATGCCCC |
| | GCCTCTCTCC | TCGT | (SEQ ID NO: 63) |
| | TCTT | (SEQ ID NO: 14) | |
| | (SEQ ID NO: 13) | | |
| VAV3 | GGTTTAAAGC | AGGAAGCTACACT | CAGGTGTCCAAATTC |
| | TCAGTTTCAG | GAGAGTTGTGA | (SEQ ID NO: 64) |
| | CGTTT | (SEQ ID NO: 16) | |
| | (SEQ ID NO: 15) | | |
| C10ORF130 | GCTGTAAGTACA | AATGAATAGTAGTG | CTCCTGCTAAAATTTC |
| | AAGTTATTTGATT | CTGCACAT CCA | (SEQ ID NO: 65) |
| | TGTAGTGT | (SEQ ID NO: 18) | |
| | (SEQ ID NO: 17) | | |
| ERBB3 | ACAGTGATAGCA | GCCCACGCCAGTA | ATGCTGGGCGGCACTT |
| | GGATTGGTAGTG A | GAGAA | (SEQ ID NO: 66) |
| | (SEQ ID NO: 19) | (SEQ ID NO: 20) | |
| C18ORF26 | AGCAGGGCCAGA | CCATTCAATAAATA | CTGCCACGGAAGTAT |
| | TAATGTTTATGAT | CTGAGCCAGGAGT | (SEQ ID NO: 67) |
| | AAT | A | |
| | (SEQ ID NO: 21) | (SEQ ID NO: 22) | |
| VAV1 | TCCTGCCCTGAG | CTTATCCTCCCAGC | CCAGCCTGAGGACCAG |
| | GTCTGA | TCTTCATCTG | (SEQ ID NO: 68) |
| | (SEQ ID NO: 23) | (SEQ ID NO: 24) | |
| MYO3B | CCATTTGATGGT | AGAATGTGACCATA | AAGGCCAGAAAATCAC |
| | CATGAGACTAAT | ACTATCGACTGAGT | (SEQ ID NO: 69) |
| | GTTATCT | (SEQ ID NO: 26) | |
| | (SEQ ID NO: 25) | | |
| ERBB4 (1) | AATGCTTATCTTT | GGATATATTTATAC | TAGGCATCCCAAGCTC |
| | CTGGTCATGAGT | ATGATAAAAACAGT | (SEQ ID NO: 70) |
| | CTT | AGTGGTTCCTAA | |
| | (SEQ ID NO: 27) | (SEQ ID NO: 28) | |
| ERBB4 (2) | CCTTGTTGCTTTT | CTTGCCCAAGATCA | CAATCAGCCCAAATTT |
| | GATACACTCTCAT | CATGTCTAGTA | (SEQ ID NO: 71) |
| | (SEQ ID NO: 29) | (SEQ ID NO: 30) | |
| COX7B2 | AGAAGTGGACAT | TTCCCACTGCACAA | CCCAGCCTGAATTAG |
| | AAGGCCTTTAGT G | GCATACA | (SEQ ID NO: 72) |
| | (SEQ ID NO: 31) | (SEQ ID NO: 32) | |
| CDH12 | CTTTTTTTTTCTA | TCAGAAATAGCATA | ATGGCAGGCACTAAAC |
| | AGGTAAAATTAGT | TGTTTTTGGAGGCT | (SEQ ID NO: 73) |
| | AACACATTTATTT | (SEQ ID NO: 34) | |
| | GGG | | |
| | (SEQ ID NO: 33) | | |
| VAV2 | CAAAAGTGACAC | GCTGTTCGTGTCG | CAGCCCGTCATTCGCA |
| | TTACCCCAATTAC | TCTCCTT | (SEQ ID NO: 74) |
| | AG | (SEQ ID NO: 36) | |
| | (SEQ ID NO: 35) | | |
| CDH1 | GACAGGGCTTTA | GCACACGCCCTGA | CCCCTCCTCCCTTCTC |
| | TGTATTAGCCAC A | GAACA | (SEQ ID NO: 75) |
| | (SEQ ID NO: 37) | (SEQ ID NO: 38) | |
| TWIST1 | GCGCTGCGGAAG | GCTTGAGGGTCTG | CCCTCGGACAAGCTG |
| | ATCATC | AATCTTGCT | (SEQ ID NO: 76) |
| | (SEQ ID NO: 39) | (SEQ ID NO: 40) | |
| TWISTNB | ACATGGGTGATG | TGTGATATTTAGTT | ATGCTGCTGGAGTATTC |
| | AACTAGAATTTGA | TTCCCCGAATGCA | (SEQ ID NO: 77) |
| | AGT | (SEQ ID NO: 42) | |
| | (SEQ ID NO: 41) | | |
| RPL15 | AGATTGGTAAGC | CGTCTAAGCTCACA | CTCACCAGCTTCCC |
| | TAGCAATGAATG CT | CTTGAAAGGTA | (SEQ ID NO: 78) |
| | (SEQ ID NO: 43) | (SEQ ID NO: 44) | |
| TFRC1 | ACTTACTACACCT | AACATTTTAAGCAC | TCTTCTTGTGTCAACTTTG |
| | GGCCATGGA | TGCAGTAAATTTGG | (SEQ ID NO: 79) |
| | (SEQ ID NO: 45) | T | |
| | | (SEQ ID NO: 46) | |
| SDK1 | TGCTGGACACTT | GAGAGGACTTCCT | CCTCCGTATACTTTCTATCCC |
| | TCACTTGGAA | AGGGAACTTAGG | (SEQ ID NO: 80) |
| | (SEQ ID NO: 47) | (SEQ ID NO: 48) | |
| GLI3 | AGTTTGGGAAGC | TCACCTTCTGATGA | CTGAGCACATTTATACAGATG |
| | CCTCCTCTAA | ACACTTTTCTGT | (SEQ ID NO: 81) |
| | (SEQ ID NO: 49) | (SEQ ID NO: 50) | |
| LANCL2 | GCCTCAGTGGGA | CATGCCTTTATTCC | CCTGCCCGCTCTGC |
| | ACTTCTGT | CAGCTTCTC | (SEQ ID NO: 82) |
| | (SEQ ID NO: 51) | (SEQ ID NO: 52) | |
| VOPP1 | AGGAAACCTTCA | CCTTGAGCAGAGA | TCACACTGGAGAGGCC |
| | GGAGCAACTC | CGTCTTTCA | (SEQ ID NO: 83) |
| | (SEQ ID NO: 53) | (SEQ ID NO: 54) | |
| NFE2L3 | GCCCCTGGTGCG | CCAAGTGCCTCAA | TTCTGTGGCAGCCAGCTG |
| | ACA | AGTTGCA | (SEQ ID NO: 84) |
| | (SEQ ID NO: 55) | (SEQ ID NO: 56) | |

### Statistical Analyses

The aCGH data were first preprocessed by averaging 10 consecutively located probes to form 36,549 disjoint blocks. A two-step statistical procedure to determine sites of amplification or deletion with high frequency of occurrence was applied. T-test to determine the DNA gain or loss status of each probe-block for each sample separately was first used and then collectively, a block as a gain-block (or loss-block) if at least 30% of the 138 samples showed gains (or losses) was claimed. To determine gain or loss status of a block, the two-sided t-test (5% significance) was used. Statistical calculation indicated that a gain/loss block claimed at 30% threshold were very unlikely to have a true prevalence less than 25% (p-value=0.0047). For comparative CNA analysis with respect to EGFR mutation status, the t-test (two-sided, 5% significance) was applied to compare two group means. Both univariate and multivariate Cox regression models were applied for prediction of patients' survival. The software, MetaCore™, was used for functional enrichment analysis. The representative CNA profile on chromosome 7p was derived by the weighted singular value decomposition method.

### Example 1

### CNA Profiling Results

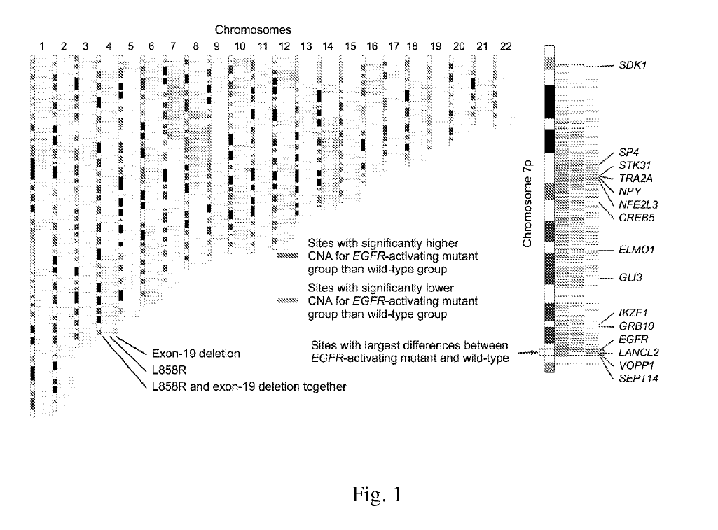

CNA profiling on 138 tumors of lung adenocarcinoma was conducted by the array CGH of NimbleGen system. The resulting CNA profiles were shown in FIG. 1A. The statistical analysis detected a total of 3,187 probe-blocks of DNA-gain and 6,029 probe-blocks of DNA-loss with false discovery rates of 0.054 and 0.028 respectively.

### Example 2

### Chromosome 7p has Highest Rate of DNA-Gain for the Gene-Harboring Regions

The chromosome sites with DNA gains were examined first. It was found that relative to the arm size, chromosome 5p, 7p, and 8q had the largest region of DNA-gain (Table 2). For the gene-harboring region, the gain rate for chromosome 7p turned out the highest. Significantly, EGFR was in the list, along with other notable genes like HDAC9, DGKB, MEOX2 and POU6F2, all of which were within the top 1% genome-wide when ranking the probe-blocks according to their average CNA values across all 138 samples (Table 3).

TABLE 2
Chromosome wide DNA gain/loss percentages

| | | | Number of | | Number of |
|---|---|---|---|---|---|
| | Number of | Number of | gene-haboring | Number of | gene-haboring |
| | probe- | gain probe- | gain probe- | loss probe- | loss probe- |
| Chromosome | blocks | blocks (%) | blocks (%) | blocks (%) | blocks (%) |
| 1p | 1624 | 144 (8.9%) | 59 (3.6%) | 316 (19.5%) | 269 (16.6%) |
| 1q | 1398 | 213 (15.2%) | 100 (7.2%) | 103 (7.4%) | 89 (6.4%) |
| 2p | 1252 | 100 (8%) | 26 (2.1%) | 148 (11.8%) | 107 (8.5%) |
| 2q | 2038 | 241 (10.5%) | 111 (5.4%) | 185 (9.1%) | 152 (7.5%) |
| 3p | 1267 | 51 (4%) | 20 (1.6%) | 213 (16.8%) | 179 (14.1%) |
| 3q | 1458 | 158 (10.8%) | 52 (3.6%) | 101 (6.9%) | 80 (5.5%) |
| 4p | 684 | 84 (12.3%) | 19 (2.8%) | 95 (13.9%) | 79 (11.5%) |
| 4q | 1910 | 253 (13.2%) | 88 (4.6%) | 36 (1.9%) | 28 (1.5%) |
| 5p | 639 | 193 (30.2%) | 56 (8.8%) | 13 (2%) | 10 (1.6%) |
| 5q | 1783 | 182 (10.2%) | 50 (2.8%) | 148 (8.3%) | 116 (6.5%) |
| 6p | 812 | 48 (5.9%) | 25 (3.1%) | 115 (14.2%) | 89 (11%) |
| 6q | 1501 | 119 (7.9%) | 47 (3.1%) | 87 (5.8%) | 66 (4.4%) |
| 7p | 783 | 213 (27.2%) | 89 (11.4%) | 52 (6.6%) | 44 (5.6%) |
| 7q | 1271 | 187 (14.7%) | 87 (6.8%) | 161 (12.7%) | 129 (10.1%) |
| 8p | 594 | 7 (1.2%) | 2 (0.3%) | 168 (28.3%) | 123 (20.7%) |
| 8q | 1393 | 275 (19.7%) | 102 (7.3%) | 69 (4.9%) | 51 (3.7%) |
| 9p | 541 | 25 (4.6%) | 9 (1.7%) | 70 (12.9%) | 61 (11.3%) |
| 9q | 975 | 17 (1.7%) | 3 (0.3%) | 307 (31.5%) | 252 (25.8%) |
| 10p | 537 | 1 (0.2%) | 0 (0%) | 96 (17.9%) | 75 (14%) |
| 10q | 1245 | 27 (2.2%) | 5 (0.4%) | 296 (23.8%) | 237 (19%) |
| 11p | 693 | 72 (10.4%) | 21 (3%) | 86 (12.4%) | 78 (11.3%) |
| 11q | 1097 | 84 (7.7%) | 38 (3.5%) | 198 (18%) | 172 (15.7%) |
| 12p | 474 | 25 (5.3%) | 16 (3.4%) | 57 (12%) | 52 (11%) |
| 12q | 1325 | 99 (7.5%) | 48 (3.6%) | 272 (20.5%) | 241 (18.2%) |
| 13q | 1354 | 85 (6.3%) | 17 (1.3%) | 147 (10.9%) | 116 (8.6%) |
| 14q | 1213 | 143 (11.8%) | 33 (2.7%) | 160 (13.2%) | 136 (11.2%) |
| 15q | 1073 | 18 (1.7%) | 9 (0.8%) | 309 (28.8%) | 244 (22.7%) |
| 16p | 406 | 3 (0.7%) | 0 (0%) | 143 (35.2%) | 131 (32.3%) |
| 16q | 614 | 12 (2%) | 2 (0.3%) | 191 (31.1%) | 160 (26.1%) |
| 17p | 277 | 0 (0%) | 0 (0%) | 191 (69%) | 163 (58.8%) |
| 17q | 725 | 22 (3%) | 6 (0.8%) | 275 (37.9%) | 247 (34.1%) |
| 18p | 204 | 2 (1%) | 1 (0.5%) | 30 (14.7%) | 24 (11.8%) |
| 18q | 864 | 45 (5.2%) | 18 (2.1%) | 121 (14%) | 89 (10.3%) |
| 19p | 287 | 3 (1%) | 0 (0%) | 210 (73.2%) | 200 (69.7%) |
| 19q | 395 | 4 (1%) | 0 (0%) | 246 (62.3%) | 229 (58%) |
| 20p | 372 | 13 (3.5%) | 6 (1.6%) | 54 (14.5%) | 49 (13.2%) |
| 20q | 468 | 1 (0.2%) | 0 (0%) | 200 (42.7%) | 163 (34.8%) |
| 21q | 469 | 45 (9.6%) | 13 (2.8%) | 82 (17.5%) | 71 (15.1%) |
| 22q | 444 | 0 (0%) | 0 (0%) | 279 (62.8%) | 245 (55.2%) |

TABLE 3
Top 1% probe blocks with highest DNA gain in 138 lung adenocarcinomas.

| Gene | Cytoband | Gene name | CNA Mean |
|---|---|---|---|
| PAPP2B | 1p32.2 | phosphatidic acid phosphatase type 2B | 0.2115 |
| LRRIQ3 | 1p31.1 | leucine-rich repeats and IQ motif containing 3 | 0.2119 |
| Cforf173 | 1p31.1 | chromosome 1 open reading frame 173 | 0.1921 |
| Cforf173 | 1p31.1 | chromosome 1 open reading frame 173 | 0.2329 |
| TTLL7 | 1p31.1 | tubulin tyrosine ligase-like family, member 7 | 0.2106 |
| PKN2 | 1p22.2 | protein kinase N2 | 0.2316 |
| PKN2 | 1p22.2 | protein kinase N2 | 0.2033 |
| SNX7 | 1p21.3 | sorting nexin 7 | 0.2149 |
| OLFM3 | 1p21.1 | olfactomedin 3 | 0.2239 |
| OLFM3 | 1p21.1 | olfactomedin 3 | 0.1962 |
| AMY2A | 1p21.1 | amylase, alpha 2A (pancreatic) | 0.1997 |
| LOC100129138 | 1p21.1 | THAP domain containing, apoptosis associated protein 3 | 0.1978 |
| | | pseudogene | |
| PRMT6 | 1p13.3 | protein arginine methyltransferase 6 | 0.2609 |
| PRMT6 | 1p12.3 | protein arginine methyltransferase 6 | 0.2221 |
| RPTN | 1q21.3 | repetin | 0.1932 |
| FLG | 1q21.3 | filaggrin | 0.2912 |
| NUF2 | 1q23.3 | NUF2, NDC80 kinetochore complex component homolog | 0.0078 |
| | | (S. cerevisiae) | |
| PBX1 | 1q23.3 | pre-B-cell leukemia homebox 1 | 0.2012 |
| DNM3 | 1q24.3 | dynamin 3 | 0.2498 |
| TNFSF18 | 1q25.1 | tumor necrosis factor (ligand) superfamily, member 18 | 0.1984 |
| TNR | 1q25.1 | tenascin R (restrictin, janusin) | 0.1914 |
| FAM5B | 1q25.2 | family with sequence similarity 5, member B | 0.2206 |
| HMCN1 | 1q31.1 | hemicentin 1 | 0.2213 |
| HMCN1 | 1q31.1 | hemicentin 1 | 0.2415 |
| TPR | 1q31.1 | translocated promoter region (to activaated MET oncogene) | 0.1943 |
| PLA2G4A | 1q31.1 | phospholipase A2, group IVA (cytosolic, calcium-dependent) | 0.1958 |
| PLA2G4A | 1q31.1 | phospholipase A2, group IVA (cytosolic, calcium-dependent) | 0.1954 |
| FAM5C | 1q31.1 | family with sequence similarity 5, memeber C | 0.2118 |
| FAM5C | 1q31.1 | family with sequence similarity 5, memeber C | 0.2207 |
| FAM5C | 1q31.1 | family with sequence similarity 5, memeber C | 0.1915 |
| LOC440704 | 1q31.2 | hypothetical LOC440704 | 0.2018 |
| RGS18 | 1q31.2 | regulator of G-protein signaling 18 | 0.1955 |
| RGS0 | 1q31.2 | regulator of G-protein signaling 21 | 0.2464 |
| CDC73 | 1q31.3 | cell division cycle 73, Paf1/RNA polymerase II | 0.235 |
| | | complex component, homolog (S. cerevisiae) | |
| KCNT2 | 1q31.3 | potassium channel, subfamily T, member 2 | 0.2064 |
| KCNT2 | 1q31.3 | potassium channel, subfamily T, member 2 | 0.2111 |
| KCNT2 | 1q31.3 | potassium channel, subfamily T, member 2 | 0.1964 |
| KCNT2 | 1q31.3 | potassium channel, subfamily T, member 2 | 0.281 |
| KCNT2 | 1q31.3 | potassium channel, subfamily T, member 2 | 0.2141 |
| KCNT2 | 1q31.3 | potassium channel, subfamily T, member 2 | 0.196 |
| CFH | 1q31.3 | complement factor H | 0.2026 |
| CRB1 | 1q31.3 | crumbs homolog 1 (Drosophila) | 0.2087 |
| CRB1 | 1q31.3 | crumbs homolog 1 (Drosophila) | 0.2639 |
| LHX9 | 1q31.3 | LIM homebox 9 | 0.1966 |
| MIR181A1 | 1q31.3 | microRNA 181a-1 | 0.2004 |
| CAMK1G | 1q32.2 | calcium/calmodulin-dependent protein kinase IG | 0.2402 |
| USH2A | 1q41 | Usher syndrome 2A (autosomal recessive, mild) | 0.2201 |
| USH2A | 1q41 | Usher syndrome 2A (autosomal recessive, mild) | 0.2072 |
| ESRRG | 1q41 | estrogen-related receptor gamma | 0.246 |
| ESRRG | 1q41 | estrogen-related receptor gamma | 0.207 |
| ESRRG | 1q41 | estrogen-related receptor gamma | 0.1996 |
| LYPLAL1 | 1q41 | lysophospholipase-like 1 | 0.215 |
| SMYD3 | 1q44 | SET and MYND domain containing 3 | 0.2474 |
| APOB | 2p24.1 | apolipoprotein B (including Ag(x) antigen) | 0.2063 |
| APOB | 2p24.1 | apolipoprotein B (including Ag(x) antigen) | 0.2287 |
| KLHL29 | 2p24.1 | kelch-like 29 (Drosophila) | 0.2171 |
| SLC8A1 | 2p22.1 | solute carrier family 8 (sodium/calcium exchanger), member 1 | 0.2023 |
| SLC8A1 | 2p22.1 | solute carrier family 8 (sodium/calcium exchanger), member 1 | 0.2092 |
| NRXN1 | 2p16.3 | neurexin 1 | 0.2886 |
| ASB3 | 2p16.2 | ankyrin repeat and SOCS box-containing 3 | 0.2016 |
| CCDC85A | 2p16.1 | coiled-coil domain containing 85A | 0.1919 |
| FLJ30638 | 2p16.1 | hypothetical LOC400955 | 0.2919 |
| LRRTM4 | 2p12 | leucine rich repeat transmembrane neuronal 4 | 0.1982 |
| CTNNA2 | 2p12 | catenin (cadherin-associated protein), alpha 2 | 0.2143 |
| DPP10 | 2q14.1 | dipeptidyl-peptidase 10 (non-functional) | 0.2505 |
| DPP10 | 2q14.1 | dipeptidyl-peptidase 10 (non-functional) | 0.1937 |
| DPP10 | 2q14.1 | dipeptidyl-peptidase 10 (non-functional) | 0.2183 |
| LRP1B | 2q22.1 | low density lipoprotein receptor-related protein 1B | 0.2224 |
| LRP1B | 2q22.2 | low density lipoprotein receptor-related protein 1B | 0.2049 |
| ARHGAP15 | 2q22.2 | Rho GTPase activating protein 15 | 0.1992 |
| DKFZp686O1327 | 2q22.3 | hypothetical LOC401014 | 0.2066 |
| KCNJ3 | 2q24.1 | potassium, inwardly-rectifying channel, subfamily J, member 3 | 0.2355 |
| DPP4 | 2q24.2 | dipeptidyl-peptidase 4 | 0.1975 |
| GRB14 | 2q24.3 | growth factor receptor-bound protein 14 | 0.2435 |
| SCN1A | 2q24.3 | sodium channel, voltage-gated, type I, alpha subunit | 0.2394 |
| XIRP2 | 2q24.3 | xin actin-binding repeat containing 2 | 0.1977 |
| TTN | 2q31.2 | titin | 0.2498 |
| UBE2E3 | 2q31.3 | ubiquitin-conjugation enzyme E2E 3(UBC4/5 homolog, yeast) | 0.2315 |
| UBE2E3 | 2q31.3 | ubiquitin-conjugation enzyme E2E 3(UBC4/5 homolog, yeast) | 0.1988 |
| ZNF804A | 2q32.1 | zinc finger protein 804A | 0.1947 |
| ZNF804A | 2q32.1 | zinc finger protein 804A | 0.1966 |
| SLC39A10 | 2q32.3 | solute carrier family 39 (zinc transporter), memeber 10 | 0.1953 |
| PLCL1 | 2q33.1 | phospholipase C-like 1 | 0.2125 |
| SATB2 | 2q33.1 | SATB homebx 2 | 0.2014 |
| ERBB4 | 2q34 | v-erb-a erythroblastic luekemia viral oncogene homolog 4 (avian) | 0.2414 |
| ZNF385D | 3p24.3 | zinc finger protein 385D | 0.2016 |
| GADL1 | 3p23 | glutamate decarboxylase-like 1 | 0.2252 |
| EPHA3 | 3p11.1 | EPH receptor A3 | 0.1977 |
| ABI3BP | 3q12.2 | ABI, member 3 (NESH) binding protein | 0.2059 |
| ZPLD1 | 3q12.3 | zona pellucida-like domain containing 1 | 0.2492 |
| ZPLD1 | 3q13.11 | zona pellucida-like domain containing 1 | 0.2067 |
| PVRL3 | 3q13.13 | poliovirus receptor-related 3 | 0.2266 |
| C3orf58 | 3q24 | chromosome 3 open reading frame 58 | 0.1935 |
| C3orf58 | 3q24 | chromosome 3 open reading frame 58 | 0.2105 |
| PLOD2 | 3q24 | procollagen-lysine, 2-oxoglutarate 5-dioxygenase 2 | 0.2748 |
| SI | 3q26.1 | sucrase-isomaltase (alpha-glucosidase) | 0.2224 |
| BCHE | 3q26.1 | butyrylcholinesterase | 0.2615 |
| LOC646168 | 3q26.1 | hypothetical protein LOC646168 | 0.2086 |
| MECOM | 3q26.2 | MDS1 and EVI1 complex locus | 0.1927 |
| FGF12 | 3q28 | fibroblast growth factor 12 | 0.2441 |
| PCDH7 | 4p15.1 | protocadherin 7 | 0.2145 |
| PCDH7 | 4p15.1 | protocadherin 7 | 0.1924 |
| ARAP2 | 4p15.1 | ArfGAP with RhoGAP domain, ankyrin repeat and PH domain 2 | 0.222 |
| ARAP2 | 4p14 | ArfGAP with RhoGAP domain, ankyrin repeat and PH domain 2 | 0.2042 |
| GNPDA2 | 4p13 | glucosamine-6-phosphate deaminase 2 | 0.1935 |
| EPHA5 | 4q13.1 | EPH receptor A5 | 0.2336 |
| ADAMTS3 | 4q13.3 | ADAM metallopeptidase with throbospondin type 1 motif, 3 | 0.2106 |
| AREG | 4q13.3 | amphiregulin | 0.1974 |
| GDEP | 4q21.21 | gene differentially expressed in prostate | 0.2004 |
| PDHA2 | 4q22.3 | pyruvate dehydrogenase (lipoamide) alpha 2 | 0.2965 |
| C4orf37 | 4q22.3 | chromosome 4 open reading frame 37 | 0.2313 |
| PITX2 | 4q25 | paired-like homeodomain 2 | 0.1995 |
| TRAM1L1 | 4q26 | translocation associated membrane protein 1-like 1 | 0.1978 |
| C4orf33 | 4q28.2 | chromosome 4 open reading frame 33 | 0.1988 |
| PCDH18 | 4q28.3 | protocadherin 18 | 0.2112 |
| GRIA2 | 4q32.1 | glutamate receptor, ionotropic, AMPA 2 | 0.2003 |
| LOC285501 | 4q34.3 | hypothetical protein LOC285501 | 0.2217 |
| LOC340094 | 5p15.32 | hypothetical LOC340094 | 0.1976 |
| LOC285692 | 5p15.2 | hypothetical LOC285692 | 0.2127 |
| CTNND2 | 5p15.2 | catenin (cadherin-associated protein), delta 2 | 0.2152 |
| | | (neural plakophilin-related arm-repeat protein) | |
| CTNND2 | 5p15.2 | catenin (cadherin-associated protein), delta 2 | 0.2293 |
| | | (neural plakophilin-related arm-repeat protein) | |
| DNAH5 | 5p15.2 | dynein, axonemal, heavy chain 5 | 0.1924 |
| DNAH5 | 5p15.2 | dynein, axonemal, heavy chain 5 | 0.2171 |
| DNAH5 | 5p15.2 | dynein, axonemal, heavy chain 5 | 0.2347 |
| DNAH5 | 5p15.2 | dynein, axonemal, heavy chain 5 | 0.1948 |
| FBXL7 | 5p15.1 | F-box and leucine-rich repeat protein 7 | 0.2802 |
| LOC401177 | 5p15.1 | hypothetical LOC401177 | 0.1976 |
| CDH18 | 5p14.3 | cadherin 18, type 2 | 0.2171 |
| CDH18 | 5p14.3 | cadherin 18, type 2 | 0.2318 |
| CDH18 | 5p14.3 | cadherin 18, type 2 | 0.212 |
| CDH18 | 5p14.3 | cadherin 18, type 2 | 0.2099 |
| CDH18 | 5p14.3 | cadherin 18, type 2 | 0.2299 |
| CDH12 | 5p14.3 | cadherin 12, type 2 (N-cadherin 2) | 0.2152 |
| CDH12 | 5p14.3 | cadherin 12, type 2 (N-cadherin 2) | 0.2502 |
| CDH12 | 5p14.3 | cadherin 12, type 2 (N-cadherin 2) | 0.2326 |
| CDH12 | 5p14.3 | cadherin 12, type 2 (N-cadherin 2) | 0.2104 |
| CDH9 | 5p14.1 | cadherin 9 type 2 (T1-cadherin 2) | 0.2323 |
| CDH9 | 5p14.1 | cadherin 9 type 2 (T1-cadherin 2) | 0.1935 |
| CDH9 | 5p14.1 | cadherin 9 type 2 (T1-cadherin 2) | 0.238 |
| CDH9 | 5p14.1 | cadherin 9 type 2 (T1-cadherin 2) | 0.1923 |
| LOC729862 | 5p14.1 | straitin, calmodulin binding protein psuedogene | 0.2219 |
| LOC729862 | 5p14.1 | straitin, calmodulin binding protein psuedogene | 0.2457 |
| LOC729862 | 5p13.3 | straitin, calmodulin binding protein psuedogene | 0.1979 |
| CDH6 | 5p13.3 | cadherin 6, type 2, K-cadherin (fetal kidney) | 0.1991 |
| CDH6 | 5p13.3 | cadherin 6, type 2, K-cadherin (fetal kidney) | 0.1968 |
| CDH6 | 5p13.3 | cadherin 6, type 2, K-cadherin (fetal kidney) | 0.2332 |
| CDH6 | 5p13.3 | cadherin 6, type 2, K-cadherin (fetal kidney) | 0.2033 |
| PLCXD3 | 5p13.1 | phosphatidylinosotol-specific phospholipase C, | 0.2054 |
| | | X domain containing 3 | |
| OXCT1 | 5p13.1 | 3-oxoacid CoA transferase 1 | 0.2194 |
| NNT | 5p12 | nicotinamide nucleotide transhydrogenase | 0.3061 |
| FGF10 | 5p12 | fibroblast growth factor 10 | 0.2095 |
| HCN1 | 5p12 | hyperpolarization activated cyclic nucleotide-gated | 0.2184 |
| | | potassium channel 1 | |
| HCN1 | 5p11 | hyperpolarization activated cyclic nucleotide-gated | 0.2354 |
| | | potassium channel 1 | |
| HCN1 | 5p11 | hyperpolarization activated cyclic nucleotide-gated | 0.2756 |
| | | potassium channel 1 | |
| HCN1 | 5p11 | hyperpolarization activated cyclic nucleotide-gated | 0.234 |
| | | potassium channel 1 | |
| HCN1 | 5p11 | hyperpolarization activated cyclic nucleotide-gated | 0.2099 |
| | | potassium channel 1 | |
| PDE4D | 5q12.1 | phosphodiesterase 4D, cAMP-specific | 0.1952 |
| MEF2C | 5q14.3 | myocyte enhancer factor 2C | 0.2164 |
| MEF2C | 5q14.3 | myocyte enhancer factor 2C | 0.2027 |
| CETN3 | 5q14.3 | centrin, EF-hand protein, 3 | 0.2484 |
| ST8SIA4 | 5q21.1 | STB alpha-N-acetyl-neuraminide alpha-2,8-sialyltransferase 4 | 0.2113 |
| EFNA5 | 5q21.3 | ephrin-A5 | 0.227 |
| FBXL17 | 5q21.3 | F-box and leucine-rich repeat protein 17 | 0.2163 |
| FAM170A | 5q23.1 | family with sequence similarity 170, member A | 0.2047 |
| KCTD16 | 5q32 | potassium channel tetramerisation domain containing 16 | 0.2027 |
| GABRG2 | 5q34 | gamma-aminobutyric acid (GABA) A receptor, gamma 2 | 0.2008 |
| MAT2B | 5q34 | methionine adenosyltransferase II, beta | 0.2047 |
| ODZ2 | 5q34 | odz, add OZ/ten-m homolog 2 (Drosophila) | 0.2264 |
| OPN5 | 6p12.3 | opsin 5 | 0.2037 |
| C6orf138 | 6p12.3 | chromosome 6 open reading frame 138 | 0.2069 |
| DEFB112 | 6p12.3 | defensin, beta 112 | 0.2432 |
| PKHD1 | 6p12.2 | polycystic kidney and hepatic disease 1 (autosamal recessive) | 0.1926 |
| MTRNR2L9 | 6q11.1 | MT-RNR2-like 9 | 0.2125 |
| EYS | 6q12 | eyes shut homolog (Drosophila) | 0.2137 |
| EPHA7 | 6q16.1 | EPH receptor A7 | 0.2043 |
| KLHL32 | 6q16.1 | kelch-like 32 (drosophila) | 0.192 |
| SDK1 | 7p22.2 | sidekick homolog 1, cell adhesion molecule (chicken) | 0.2213 |
| NXPH1 | 7p21.3 | neurexophilin 1 | 0.2178 |
| NXPH1 | 7p21.3 | neurexophilin 1 | 0.198 |
| NXPH1 | 7p21.3 | neurexophilin 1 | 0.2029 |
| NXPH1 | 7p21.3 | neurexophilin 1 | 0.3486 |
| NXPH1 | 7p21.3 | neurexophilin 1 | 0.205 |
| PER4 | 7p21.3 | period homolog 3, (Drosophila) pseudogene | 0.2273 |
| PER4 | 7p21.3 | period homolog 3, (Drosophila) pseudogene | 0.2097 |
| PER4 | 7p21.3 | period homolog 3, (Drosophila) pseudogene | 0.2 |
| THSD7A | 7p21.3 | thrombospondin, type I, domain containing 7A | 0.2272 |
| THSD7A | 7p21.3 | thrombospondin, type I, domain containing 7A | 0.2872 |
| THSD7A | 7p21.3 | thrombospondin, type I, domain containing 7A | 0.2625 |
| TMEM106B | 7p21.3 | transmembrane protein 106B | 0.209 |
| ETV1 | 7p21.2 | ets variant 1 | 0.2215 |
| DGKB | 7p21.2 | diacylglycerol kinase, beta 90 kDa | 0.2607 |
| DGKB | 7p21.2 | diacylglycerol kinase, beta 90 kDa | 0.2236 |
| DGKB | 7p21.2 | diacylglycerol kinase, beta 90 kDa | 0.2292 |
| DGKB | 7p21.2 | diacylglycerol kinase, beta 90 kDa | 0.2664 |
| TMEM195 | 7p21.1 | transmembrane protein 195 | 0.2006 |
| TMEM195 | 7p21.1 | transmembrane protein 195 | 0.2133 |
| MEOX | 7p21.1 | mesenchyme homebox 2 | 0.2331 |
| MEOX | 7p21.1 | mesenchyme homebox 2 | 0.2088 |
| ISPD | 7p21.1 | isoprenoid synthase domain containing | 0.2459 |
| SNX13 | 7p21.1 | sorting nexin 13 | 0.1962 |
| PRPS1L1 | 7p21.1 | phosphoribosyl pyrophosphate synthetase 1-like 1 | 0.21 |
| HDAC9 | 7p21.1 | histone deacetylase 9 | 0.2909 |
| HDAC9 | 7p21.1 | histone deacetylase 9 | 0.1928 |
| HDAC9 | 7p21.1 | histone deacetylase 9 | 0.2498 |
| HDAC9 | 7p21.1 | histone deacetylase 9 | 0.3529 |
| HDAC9 | 7p21.1 | histone deacetylase 9 | 0.1975 |
| HDAC9 | 7p21.1 | histone deacetylase 9 | 0.2718 |
| HDAC9 | 7p21.1 | histone deacetylase 9 | 0.2003 |
| FERD3L | 7p21.1 | Fer3-like (Drosophila) | 0.1966 |
| TWISTNB | 7p15.3 | TWIST neighbor | 0.268 |
| RPL23P8 | 7p15.3 | ribosomal protein L23 pseudogene 8 | 0.1998 |
| NPVF | 7p15.2 | neuropeptide VF precursor | 0.1949 |
| MIR148A | 7p15.2 | microRCA 148a | 0.1961 |
| CCDC129 | 7p15.1 | coiled-coil domain containing 129 | 0.2241 |
| PDE1C | 7p15.1 | phosphodiesterase 1C, calmodulin-dependent 70 kDa | 0.2017 |
| BBS9 | 7p14.3 | Bardet-Biedl syndrome 9 | 0.2467 |
| POU6F2 | 7p14.1 | POU class 6 homebox 2 | 0.2422 |
| C7orf10 | 7p14.1 | chromosome 7 open reading frame 10 | 0.2008 |
| ABCA13 | 7p12.3 | ATP-binding cassette, sub-family A (ABC1), member 3 | 0.2018 |
| CDC14C | 7p12.3 | CDC 14 cell division cycle 14 homolog C (S. cerevisiae) | 0.1977 |
| CDC14C | 7p12.3 | CDC 14 cell division cycle 14 homolog C (S. cerevisiae) | 0.2338 |
| VWC2 | 7p12.3 | von Willebrand factor C domain containing 2 | 0.2143 |
| POM121L12 | 7p12.1 | POM121 membrane glycoprotein-like 12 | 0.1945 |
| HPVC1 | 7p11.2 | human papillomavirus (tpe 18) E5 central sequence-like 1 | 0.1979 |
| HPVC1 | 7p11.2 | human papillomavirus (tpe 18) E5 central sequence-like 1 | 0.1944 |
| EGFR | 7p11.2 | epidermal growth factor receptor | 0.216 |
| LOC642006 | 7p11.2 | glucuronidase, beta pseudogene | 0.2114 |
| ZNF716 | 7p11.1 | zinc finger protein 716 | 0.2088 |
| LOC643955 | 7q11.21 | zinc finger protein 479 pseudogene | 0.2005 |
| LOC643955 | 7q11.21 | zinc finger protein 479 pseudogene | 0.3332 |
| LOC643955 | 7q11.21 | zinc finger protein 479 pseudogene | 0.215 |
| LOC643955 | 7q11.21 | zinc finger protein 479 pseudogene | 0.2105 |
| LOC643955 | 7q11.21 | zinc finger protein 479 pseudogene | 0.3048 |
| LOC643955 | 7q11.21 | zinc finger protein 479 pseudogene | 0.2315 |
| SEMA3D | 7q21.11 | sema domain, immunoglobulin domain (Ig), short basic | 0.1987 |
| | | domain, secreted, (semaphorin) 3D | |
| SEMA3D | 7q21.11 | sema domain, immunoglobulin domain (Ig), short basic | 0.2551 |
| | | domain, secreted, (semaphorin) 3D | |
| SEMA3D | 7q21.11 | sema domain, immunoglobulin domain (Ig), short basic | 0.2206 |
| | | domain, secreted, (semaphorin) 3D | |
| GRM3 | 7q21.11 | glutamate receptor. metabotropic 3 | 0.2226 |
| GRM3 | 7q21.11 | glutamate receptor. metabotropic 3 | 0.229 |
| DMTF1 | 7q21.12 | cyclin D binding ,yb-like transcription factor 1 | 0.2539 |
| ZNF804B | 7q21.13 | zinc finger protein 804B | 0.2178 |
| CCDC132 | 7q21.3 | coiled-coil domain containing 132 | 0.2172 |
| CALCR | 7q21.3 | calcitonin receptor | 0.2362 |
| CALCR | 7q21.3 | calcitonin receptor | 0.1957 |
| PPP1R3A | 7q31.1 | protein phosphatase 1, regulatory (inhibitor) subunit 3A | 0.1918 |
| FOXP2 | 7q31.1 | forkhead box P2 | 0.1946 |
| TFEC | 7q31.2 | ttranscription factor EC | 0.2079 |
| TES | 7q31.2 | testis derived transcript (3 LIM domains) | 0.2985 |
| KCND2 | 7q31.31 | potassium voltage-gated channel, Shal-related subfamily, | 0.2694 |
| | | member 2 | |
| C7orf58 | 7q31.31 | chromosome 7 open reading frame 58 | 0.235 |
| GRM8 | 7q31.33 | glutamate receptor, metabotropic 8 | 0.1918 |
| POTEA | 8p11.1 | POTE ankyrin domain family, member A | 0.2482 |
| POTEA | 8p11.1 | POTE ankyrin domain family, member A | 0.2804 |
| POTEA | 8p11.1 | POTE ankyrin domain family, member A | 0.1915 |
| YTHDF3 | 8q12.3 | YTH doamin family, member 3 | 0.2641 |
| LOC100130155 | 8q12.3 | hypothetical LOC100130155 | 0.2114 |
| CC8orf34 | 8q13.2 | chromosome 8 open reading frame 34 | 0.1994 |
| ZFHX4 | 8q.2111 | zinc finger homebox 4 | 0.256 |
| PEX2 | 8q21.12 | peroxisomal biogenesis factor 2 | 0.2401 |
| PKIA | 8q21.12 | protein kinase (cAMP-dependent, catalytic) inhibitor alpha | 0.2117 |
| SNX16 | 8q21.13 | sorting nexin 16 | 0.1929 |
| CALB1 | 8q21.3 | calbindin 1, 28 kDa | 0.2122 |
| C8orf83 | 8q22.1 | chromosome 8 open reading frame 83 | 0.1918 |
| PGCP | 8q22.1 | plasma glutamate carboxypeptidase | 0.2947 |
| ZFPM2 | 8q22.3 | zinc finger protein, multitype 2 | 0.2272 |
| ZFPM2 | 8q23.1 | zinc finger protein, multitype 2 | 0.3113 |
| SYBU | 8q23.2 | syntabulin (syntaxin-interacting) | 0.2134 |
| CSMD3 | 8q23.3 | CUB and Sushi multiple domains 3 | 0.1922 |
| CSMD3 | 8q23.3 | CUB and Sushi multiple domains 3 | 0.2235 |
| CSMD3 | 8q23.3 | CUB and Sushi multiple domains 3 | 0.1952 |
| CSMD3 | 8q23.3 | CUB and Sushi multiple domains 3 | 0.2199 |
| CSMD3 | 8q23.3 | CUB and Sushi multiple domains 3 | 0.3086 |
| CSMD3 | 8q23.3 | CUB and Sushi multiple domains 3 | 0.2099 |
| CSMD3 | 8q23.3 | CUB and Sushi multiple domains 3 | 0.2081 |
| CSMD3 | 8q23.3 | CUB and Sushi multiple domains 3 | 0.2069 |
| CSMD3 | 8q23.3 | CUB and Sushi multiple domains 3 | 0.1958 |
| CSMD3 | 8q23.3 | CUB and Sushi multiple domains 3 | 0.2039 |
| TRPS1 | 8q23.3 | trichorhinophalangeal syndrome I | 0.1925 |
| SLC30A8 | 8q24.11 | solute carrier family 30 (zinc transporter), member 8 | 0.258 |
| SAMD12 | 8q24.12 | sterile alpha motif domain containing 12 | 0.2635 |
| MRPL13 | 8q24.12 | mitochondrial ribosomal protein L13 | 0.2103 |
| POU5F1B | 8q24.21 | POU class 5 homebox 1B | 0.2059 |
| MIR1208 | 8q24.21 | mircoRNA 1208 | 0.2022 |
| MIR1208 | 8q24.21 | mircoRNA 1208 | 0.3121 |
| LOC728724 | 8q24.21 | hCG1814486 | 0.1974 |
| ASAP1 | 8q24.21 | ArfGAP with SH3 domain, ankyrin repeat and PH domain 1 | 0.2033 |
| ADCY8 | 8q24.22 | adenylate cyclase 8 (brain) | 0.2013 |
| KHDRBS3 | 8q24.23 | KH doamin containing, RNA binding, signal transduction | 0.1966 |
| | | associated 3 | |
| FAM135B | 8q24.23 | family with sequence similarity 135, member 8 | 0.1976 |
| JAK2 | 9p24.1 | Janus kinase 2 | 0.3072 |
| LINGO2 | 9p21.2 | leucine rich repeat and Ig domain containing 2 | 0.1927 |
| NELL1 | 11p15.1 | NEL-like 1 (chicken) | 0.2098 |
| NELL1 | 11p15.1 | NEL-like 1 (chicken) | 0.2512 |
| KCNA4 | 11p14.1 | potassium voltage-gated channel. shaker-related subfamily, | 0.2045 |
| | | member 4 | |
| OR4A47 | 11p11.2 | olfactory receptor, family 4, subfamily A, member 47 | 0.2163 |
| LOC646813 | 11p11.12 | DEAH (Asp-Glu-Ala-His) box polypeptide 9 pseudogene | 0.2223 |
| LOC646813 | 11p11.12 | DEAH (Asp-Glu-Ala-His) box polypeptide 9 pseudogene | 0.2723 |
| OR4A5 | 11p11.12 | olfactory receptor, family 4, subfamily A, member 8 | 0.2231 |
| OR8U8 | 11q11 | olfactory receptor, family 8, subfamily U, member 5 | 0.2135 |
| MIR4300 | 11q14.1 | microRNA 4300 | 0.2378 |
| RAB38 | 11q14.2 | RAB38, member RAS oncogene family | 0.2297 |
| GRIA4 | 11q22.3 | glutamate receptor, ionotrophis, AMPA 4 | 0.2141 |
| MGST1 | 12p12.3 | microsomal glutathione S-transferase 1 | 0.1928 |
| PLCZ1 | 12p12.3 | phospholipase C, zeta 1 | 0.1966 |
| ABCC9 | 12p12.1 | ATP-binding cassette, sub-family C (CFTR/MRP), member 9 | 0.3254 |
| SOX5 | 12p12.1 | SRY (sex determining region Y)-box 5 | 0.232 |
| SOX5 | 12p12.1 | SRY (sex determining region Y)-box 5 | 0.2355 |
| ALG10B | 12q11 | asparagine-linked glycosylation 10, alpha-1,2-glucosyltransferase | 0.1953 |
| | | homolog B (yeast) | |
| ALG10B | 12q12 | asparagine-linked glycosylation 10, alpha-1,2-glucosyltransferase | 0.2311 |
| | | homolog B (yeast) | |
| LRRK2 | 12q12 | leucine-rich repeat kinase 2 | 0.192 |
| FAM19A2 | 12q14.1 | family with sequence similarity 19 (chemokine (C-C motif)-like), | 0.2478 |
| | | member A2 | |
| LOC283392 | 12q21.1 | hypothetical LOC283392 | 0.1985 |
| TRHDE | 12q21.1 | thyrotropin-releasing hormone degrading enzyme | 0.1973 |
| PPFIA2 | 12q21.31 | protein tyrosine phosphatase, receptor type, f polypeptide (PTPRF), | 0.204 |
| | | interacting protein (lipirin), alpha 2 | |
| EPYC | 12q21.33 | epiphycan | 0.2103 |
| PRR20A | 13q21.1 | proline rich 20A | 0.3125 |
| PCDH9 | 13q21.32 | protocadherin 9 | 0.1972 |
| GPC6 | 13q31.3 | glypican 6 | 0.193 |
| OR4E2 | 14q11.2 | olfactory receptor, family 4, subfamily E, member 2 | 0.1944 |
| OR4E2 | 14q11.2 | olfactory receptor, family 4, subfamily E, member 2 | 0.2203 |
| NOVA1 | 14q12 | neuro-oncological ventral antigen 1 | 0.2722 |
| NOVA1 | 14q12 | neuro-oncological ventral antigen 1 | 0.1931 |
| MIR4307 | 14q12 | microRNA 4307 | 0.2268 |
| PRKD1 | 14q12 | protein kinase D1 | 0.239 |
| PRKD1 | 14q12 | protein kinase D1 | 0.2057 |
| PRKD1 | 14q12 | protein kinase D1 | 0.1934 |
| AKAP6 | 14q13.1 | A kinase (PRKA) anchor protein 6 | 0.22 |
| NPAS3 | 14q13.1 | neuronal PAS domain protein 3 | 0.2346 |
| NPAS3 | 14q13.1 | neuronal PAS domain protein 3 | 0.1919 |
| NPAS3 | 14q13.1 | neuronal PAS domain protein 3 | 0.1913 |
| NPAS3 | 14q13.1 | neuronal PAS domain protein 3 | 0.2236 |
| MBIP | 14q13.3 | MAP3K12 binding inhibitory protein 1 | 0.2078 |
| MBIP | 14q13.3 | MAP3K12 binding inhibitory protein 1 | 0.2042 |
| SLC25A21 | 14q13.3 | solute carrier family 25 (mitochondrial oxodicarboxylate carrier), | 0.2344 |
| | | member 21 | |
| FOXA1 | 14q21.1 | forkhead box A1 | 0.2183 |
| SEC23A | 14q21.1 | Sec23 homolog A (S. cerevisiae) | 0.2121 |
| FBXO33 | 14q21.1 | F-box protein 33 | 0.2059 |
| FBXO33 | 14q21.1 | F-box protein 33 | 0.1988 |
| LRFN5 | 14q21.2 | leucine rich repeat and fibronectin type III domain containing 5 | 0.2004 |
| LRFN5 | 14q21.2 | leucine rich repeat and fibronectin type III domain containing 5 | 0.2475 |
| C14orf106 | 14q21.3 | chromosome 14 open reading frame 106 | 0.2477 |
| MDGA2 | 14q21.3 | MAM domain containing glycosylphosphatidylinosotol | 0.2836 |
| | | anchor 2 | |
| RPS29 | 14q22.1 | ribosomal protein S29 | 0.1915 |
| FLRT2 | 14q31.3 | fibronectin leucine rich transmembrane protein 2 | 0.1917 |
| GALC | 14q31.3 | galactosylceramidase | 0.2127 |
| LOC727924 | 15q11.2 | hypothetical LOC727924 | 0.2004 |
| LOC390705 | 16p11.2 | protein phosphoatase 2, regulatory subunit B″, beta pseudogene | 0.2208 |
| CDH8 | 16q21 | cadherin 8, type 2 | 0.2022 |
| CDH8 | 16q21 | cadherin 8, type 2 | 0.2014 |
| CA10 | 17q21.33 | carbonic anhydrase X | 0.2633 |
| KIF2B | 17q22 | kinesin family member 2B | 0.2584 |
| KIF2B | 17q22 | kinesin family member 2B | 0.2473 |
| CDH2 | 18q12.1 | cadherin 2, type 1, N-cadherin (neuronal) | 0.2013 |
| DSC3 | 18q12.1 | desmocollin 3 | 0.2018 |
| ASXL3 | 18q12.1 | additional sex combs like 3 (Drosophila) | 0.2644 |
| LOC100101266 | 19p12 | hepatitis A virus cellular receptor 1 pseudogene | 0.1988 |
| LOC10101266 | 19p12 | hepatitis A virus cellular receptor 1 pseudogene | 0.2313 |
| LOC148189 | 19q12 | hypothetical LOC148189 | 0.1955 |
| LOC148189 | 19q12 | hypothetical LOC148189 | 0.2084 |
| LOC148189 | 19q12 | hypothetical LOC148189 | 0.2196 |
| JAG1 | 20p12.2 | jagged 1 | 0.2034 |
| JAG1 | 20p12.2 | jagged 1 | 0.1977 |
| TPTE | 21p11.2 | transmembrane phosphatase with tensin homology | 0.2796 |
| TMPRSS15 | 21q21.1 | transmembrane protease, serine 15 | 0.203 |

Interestingly, among the four members of the ERBB family, while EGFR and ERBB4 (2q34) have DNA-gain, ERBB2 (17q12) and ERBB3 (12q13.2) have DNA-loss. Such gain-loss disparity was also observed in several other families such as the mesenchyme homeobox genes, MEOX2 (7p21.1, gain) and MEOX1 (17p21, loss); VAV family, VAV1 (19p13.2, loss), VAV2 (9q34.2, loss) and VAV3 (1p13.3, gain) (Table 4). To confirm the high density array CGH results, genomic real time qPCR was conducted and the DNA gain/loss pattern for several genes was validated (EGFR, ERBB4, MEOX2, TWIST1, TWISTNB, DGKB, VAV3, CDH12 from the DNA-gain list and ERBB2, ERBB3, MEOX1, CDH1, VAV1, VAV2, ACTN4, FAM102A from the DNA-loss list).

TABLE 4
Summary of DNA Gain/Loss on selected gene families

| | | Number | Number |
|---|---|---|---|
| | Number | of gain | of loss |
| Protein family | of genes | genes | genes |
| ATP-binding cassette | 41 | 4 | 21 |
| aconitase | 2 | 1 | 1 |
| acyl-CoA thioesterase | 5 | 0 | 3 |
| acyl-Conenzyme A oxidase(acyl-CoA oxidase) | 2 | 0 | 2 |
| adenylate cyclase | 8 | 2 | 3 |
| aldehyde dehydrogenase | 14 | 0 | 6 |
| ankyrin(ANK) | 3 | 2 | 1 |
| adaptor-related protein complex | 18 | 0 | 10 |
| apolipoprotein | 10 | 1 | 7 |
| ATPase | 48 | 1 | 24 |
| calcium channel, voltage-dependent(CACNA) | 21 | 2 | 14 |
| calcium/calmodulin-dependent protein kinase | 9 | 2 | 5 |
| calpaio | 10 | 0 | 6 |
| cadherin | 21 | 11 | 8 |
| chloride channel(CCLA, CLCN) | 9 | 0 | 4 |
| contactin | 6 | 4 | 1 |
| collagen | 41 | 7 | 15 |
| EPH receptor | 13 | 4 | 5 |
| ERBB Family | 4 | 2 | 2 |
| fibroblast growth factor | 12 | 3 | 5 |
| fibroblast growth factor receptor | 4 | 0 | 2 |
| glutamate receptor, metabotropic | 7 | 5 | 1 |
| integrin | 24 | 4 | 11 |
| laminin | 12 | 1 | 4 |
| mesenchyme homeobox | 2 | 1 | 1 |
| procadherin | 13 | 6 | 3 |
| phosphoinositide-3-kinase | 14 | 1 | 5 |
| ribosomal protein | 22 | 3 | 10 |
| testis expressed gene | 7 | 0 | 5 |
| tropomodulin | 3 | 0 | 1 |
| transmembrane protease | 12 | 1 | 4 |
| tumor necrosis factor receptor superfamily | 11 | 1 | 9 |
| tetraspanin | 15 | 2 | 9 |
| tubulin tyrosine ligase-like family | 13 | 1 | 8 |
| tubulin | 12 | 0 | 7 |
| UDP glucuronosyltransferase | 9 | 6 | 0 |
| guanine nucleotide exchange factory (VAV) | 3 | 1 | 2 |
| wingless-type MMTV integration site family | 13 | 0 | 9 |
| tyrosine 3-monooxygenase/tryptophan 5-monooxygenase | 5 | 0 | 4 |
| activation protein | | | |

### Example 3

### Chromosome 7p Contains the Highest Proportion of the Most Notable Amplification for the EGFR-Mutation Group

EGFR-mutation testing for the presence of the exon-21 L858R point mutation or the exon-19 in-frame deletion was conducted. It was found a total of 81 patients had EGFR mutation of either type and 57 patients were the wild-type. The sites with most notable amplification or deletion in the EGFR-mutant patients was studied and regions with highest DNA gain and loss was located by identifying the top 1% of probe-blocks with the largest and the smallest mean values of CNA respectively. Interestingly, 81 out of 364 (22.3%) probe-blocks with highest CNA fall on chromosome 7p, outnumbering all other chromosome arms despite the smallness in the arm size (carrying only 2.25% of probe blocks in the array). The same pattern still occurred for the two EGFR mutation types studied separately. On the other hand, for the EGFR wild-type group, the pattern was completely different and chromosome 7p became insignificant.

### Example 4

### Chromosome 7p is Most Enriched in Containing Sites of Differential CNA Between the EGFR-Activating Mutation Group and the EGFR Wild-Type Group

The sites of significant differences between the mutation group and the wild-type group by t-test were located (FIG. 1). It was found that among all chromosomes, the greatest CNA difference is located on the chromosome 7p. Indeed, 47.13% of the probes located in this chromosome arm have CNA differences, far exceeding the percentage (20.20%) of chromosome 14q which is at the second place. For chromosome arms with higher rate of loss-blocks (17p, 19p and 19q), only very few probe sites show significant differences, indicating that the pattern of DNA loss between the mutation group and the wild-type group is more consistent.

The size of the difference (absolute value of the mean of the mutation group minus the mean of the wild type group) was also examined. It was found the largest differences to occur on a segment (869K bps of length) at 7p112 harboring EGFR, LANCL2, VOPP1, and others. The CNA values in this region are higher for the mutation group (Table 5). Co-amplification of LANCL2 with EGFR and VOPP1 with EGFR was previously reported only in some tumors (Lu Z, et al: Glioblastoma proto-oncogene SEC61gamma is required for tumor cell survival and response to endoplasmic reticulum stress. Cancer Res 69:9105-11, 2009).

TABLE 5
The genome-wide top 20 sites with the largest effect size*.

| | | EGFR-mutant | EGFR wild-type | | |
|---|---|---|---|---|---|
| | | patients | patients | Group mean | |
| Gene | Location | (mean ± SD) | (mean ± SD) | difference | p value** |
| EGFR | 7p12 | 0.278 ± 0.348 | 0.128 ± 0.152 | 0.149 | 0.001 |
| EGFR | 7p12 | 0.239 ± 0.341 | 0.098 ± 0.151 | 0.142 | 0.001 |
| LANCL2 | 7p11.2 | 0.072 ± 0.276 | −0.065 ± 0.087 | 0.138 | 5.6E−05 |
| EGFR | 7p12 | 0.182 ± 0.329 | 0.055 ± 0.106 | 0.127 | 0.002 |
| VSTM2A | 7p11.2 | 0.200 ± 0.232 | 0.084 ± 0.095 | 0.116 | 1.0E−04 |
| VOPP1 | 7p11.2 | 0.037 ± 0.247 | −0.072 ± 0.114 | 0.109 | 0.001 |
| LANCL2 | 7p11.2 | −0.003 ± 0.263 | −0.111 ± 0.107 | 0.108 | 0.001 |
| SEPT14 | 7p11.2 | 0.034 ± 0.180 | −0.066 ± 0.096 | 0.100 | 7.8E−05 |
| VOPP1 | 7p11.2 | 0.041 ± 0.306 | −0.057 ± 0.129 | 0.098 | 0.011 |
| SEC61G | 7p11.2 | 0.112 ± 0.273 | 0.014 ± 0.089 | 0.098 | 0.003 |
| MIR148A | 7p15.2 | 0.093 ± 0.159 | −0.003 ± 0.093 | 0.096 | 1.9E−05 |
| EGFR | 7p12 | 0.168 ± 0.254 | 0.074 ± 0.096 | 0.094 | 0.003 |
| EGFR | 7p12 | 0.119 ± 0.358 | 0.029 ± 0.155 | 0.091 | 0.045 |
| C7orf10 | 7p14.1 | 0.094 ± 0.124 | 0.005 ± 0.097 | 0.090 | 5.0E−06 |
| ANK1 | 8p11.1 | −0.157 ± 0.134 | −0.068 ± 0.233 | −0.088 | 0.012 |
| ADAM3A | 8p11.23 | 0.025 ± 0.126 | 0.113 ± 0.134 | −0.088 | 1.7E−04 |
| OSBPL3 | 7p15 | 0.200 ± 0.162 | 0.115 ± 0.100 | 0.085 | 2.2E−04 |
| IKZF1 | 7p13-p11.1 | 0.119 ± 0.164 | 0.034 ± 0.139 | 0.085 | 0.001 |
| KIAA0146 | 8q11.21 | −0.047 ± 0.108 | 0.038 ± 0.117 | −0.085 | 3.2E−05 |
| SEMA5A | 5p15.2 | 0.117 ± 0.151 | 0.035 ± 0.126 | 0.082 | 0.001 |

*Effect size is the absolute value of the mean difference between EGFR-mutant group and EGFR wild-type group
** p value is calculated by t-test.

The difference between L858R mutation and the wild-type was further compared. It was found out that chromosome 7p still was the richest arm in containing sites of differences in CNA values (Table 6).

TABLE 6
Distribution of the sites showing significantly different CAN in EGFR mutation status
comparison by t-test at 5% level

| | | | | | Number of |
|---|---|---|---|---|---|
| | | Number of | Number of | Number of | differential |
| | | differential | differential | differential | probes |
| | | probes | probes | probes | between |
| | | between | between | between | L858R |
| | Total | EGFR- | L858R | exon-19 | mutant and |
| | number | mutant and | mutant and | deletion and | exon-19 |
| Chromosome | of probes | wild-type (%) | wild-type (%) | wild- type (%) | deletion (%) |
| 1p | 1624 | 125 (7.7%) | 124 (7.64%) | 54 (3.33%) | 31 (1.91%) |
| 1q | 1398 | 34 (2.43%) | 30 (2.15%) | 37 (2.65%) | 40 (2.86%) |
| 2p | 1252 | 64 (5.11%) | 60 (4.79%) | 112 (8.95%) | 84 (6.71%) |
| 2q | 2038 | 74 (3.63%) | 72 (3.53%) | 115 (5.64%) | 76 (3.73%) |
| 3p | 1267 | 63 (4.97%) | 36 (2.84%) | 118 (9.31%) | 59 (4.66%) |
| 3q | 1458 | 38 (2.61%) | 51 (3.5%) | 28 (1.92%) | 49 (3.36%) |
| 4p | 684 | 46 (6.73%) | 41 (5.99%) | 35 (5.12%) | 13 (1.9%) |
| 4q | 1710 | 121 (6.34%) | 83 (4.35%) | 162 (8.48%) | 69 (3.61%) |
| 5p | 639 | 31 (4.85%) | 10 (1.56%) | 16 (2.5%) | 10 (1.56%) |
| 5q | 1783 | 40 (2.24%) | 45 (2.52%) | 38 (2.13%) | 52 (2.92%) |
| 6p | 812 | 72 (8.87%) | 58 (7.14%) | 49 (6.03%) | 13 (1.6%) |
| 6q | 1501 | 164 (10.93%) | 140 (9.33%) | 118 (7.86%) | 35 (2.33%) |
| 7p | 783 | 369 (47.13%) | 252 (32.18%) | 146 (18.65%) | 4 (0.51%) |
| 7q | 1271 | 27 (2.12%) | 40 (3.15%) | 20 (1.57%) | 20 (1.57%) |
| 8p | 594 | 79 (13.3%) | 74 (12.46%) | 41 (6.9%) | 8 (1.35%) |
| 8q | 1393 | 203 (14.57%) | 153 (10.98%) | 134 (9.62%) | 28 (2.01%) |
| 9p | 541 | 12 (2.22%) | 16 (2.96%) | 31 (5.73%) | 23 (4.25%) |
| 9q | 975 | 55 (5.64%) | 50 (5.13%) | 55 (5.64%) | 30 (3.08%) |
| 10p | 537 | 98 (18.25%) | 74 (13.78%) | 64 (11.92%) | 16 (2.98%) |
| 10q | 1245 | 166 (13.33%) | 120 (9.64%) | 146 (11.76%) | 39 (3.13%) |
| 11p | 693 | 18 (2.6%) | 36 (5.19%) | 13 (1.88%) | 52 (7.5%) |
| 11q | 1097 | 30 (2.73%) | 32 (2.92%) | 34 (3.1%) | 50 (4.56%) |
| 12p | 474 | 68 (14.35%) | 40 (8.44%) | 66 (13.92%) | 7 (1.48%) |
| 12q | 1325 | 137 (10.34%) | 104 (7.85%) | 97 (7.32%) | 27 (2.04%) |
| 13q | 1354 | 51 (3.77%) | 30 (2.22%) | 120 (8.86%) | 112 (0.27%) |
| 14q | 1213 | 245 (20.2%) | 105 (8.66%) | 331 (27.29%) | 106 (8.74%) |
| 15q | 1073 | 54 (5.03%) | 31 (2.89%) | 132 (12.3%) | 108 (10.07%) |
| 16p | 406 | 64 (15.76%) | 39 (9.61%) | 61 (15.02%) | 17 (4.19%) |
| 16q | 614 | 12 (1.95%) | 34 (3.91%) | 8 (1.3%) | 36 (5.86%) |
| 17p | 277 | 5 (1.81%) | 9 (3.25%) | 1 (0.36%) | 5 (1.81%) |
| 17q | 725 | 9 (1.24%) | 13 (1.79%) | 12 (1.66%) | 16 (2.21%) |
| 18p | 204 | 5 (2.45%) | 8 (3.92%) | 5 (2.45%) | 16 (7.84%) |
| 18q | 864 | 28 (3.24%) | 19 (2.2%) | 106 (12.27%) | 17 (12.38%) |
| 19p | 287 | 1 (0.35%) | 2 (0.7%) | 4 (1.39%) | 9 (3.14%) |
| 19q | 395 | 4 (1.01%) | 6 (1.52%) | 3 (0.76%) | 5 (1.27%) |
| 20p | 372 | 10 (2.69%) | 19 (5.11%) | 8 (2.15%) | 20 (5.38%) |
| 20q | 468 | 8 (1.71%) | 6 (1.28%) | 15 (3.21%) | 8 (1.71%) |
| 21q | 469 | 26 (5.54%) | 15 (3.2%) | 46 (9.81%) | 22 (4.69%) |
| 22q | 444 | 3 (0.68%) | 7 (1.58%) | 11 (2.48%) | 38 (8.56%) |

The deletion group was also compared with the wild-type. Chromosome 7p was ranked second only to chromosome 14q. Lastly, we compared the differences between the deletion group and the 1,858R mutation group. The total number of significant probe-blocks was smaller, suggesting more similarity for these two mutation types. The similarity is most pronounced at chromosome 7p which has the least proportion of significant probe-blocks.

### Example 5

### Distinct Patterns of CNA Profiles on Chromosome 7p

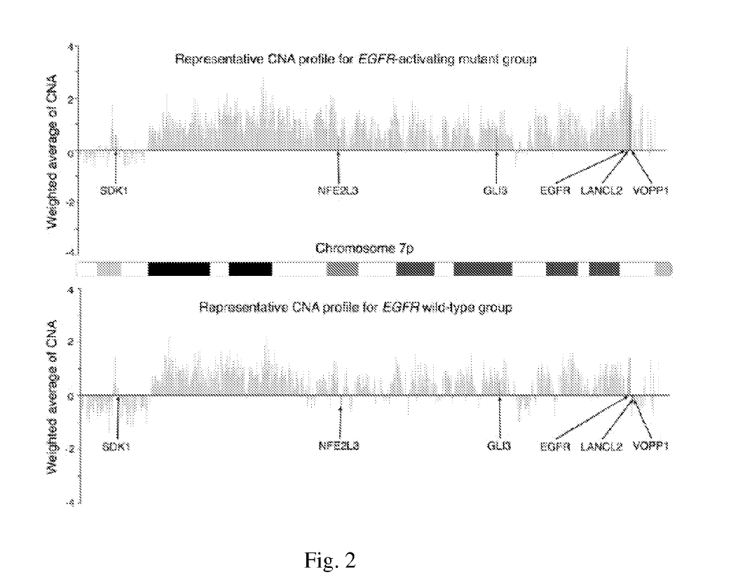

A representative CNA profile on chromosome 7p was derived for the EGFR-activating mutation group and wild-type group separately (FIG. 2). Notable differences were observed. The profile for EGFR mutation group shows consistent gains across most positions on chromosome 7p except for the beginning part of 7p22.1. On the other hand, the profile for the wild-type group shows more positions of loss and the CNA values vary considerably across chromosome 7p.

### Example 6

### Clustered Genomic Alterations in Chromosome 7p Predict Clinical Outcomes of Lung Adenocarcinoma with EGFR-Activating Mutation

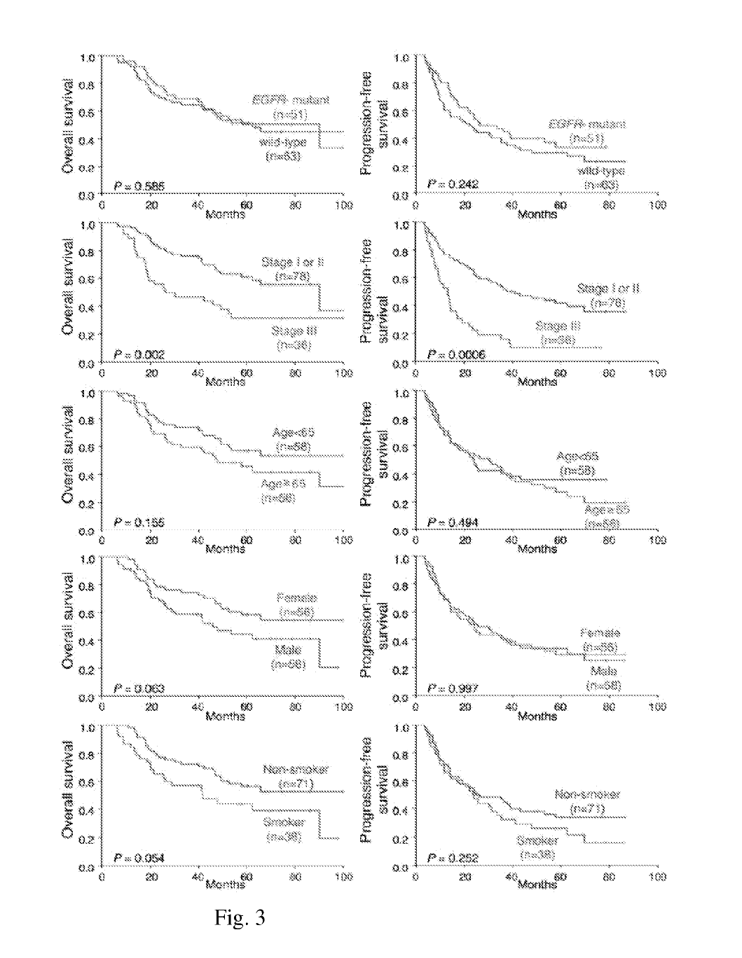

An independent group of 114 adenocarcinoma patients was collected for testing the clinical relevance of the detected genetic aberrations on chromosome 7p. After genotyping for EGFR mutation status, it was found 51 patients with EGFR-activating mutations and 63 wild-type patients. Kaplan-Meier analysis on overall survival and progression-free survivals shows a strong stage effect, but no mutation status effect (FIG. 3).

Probes for a set of six representative genes, GLI3, NFE2L3, SDK1, EGFR, VOPP1 and LANCL2, from chromosome 7p were designed and genomic real-time qPCR was conducted to measure CNAs of these genes in the 114 tumors. As discussed earlier, VOPP1 and LANCL2 are located next to EGFR. The other three genes are approximately even-spaced to cover other parts of chromosome 7p. All six genes harbor sites of differential CNA values between the EGFR mutation and wild-type from our array CGH data of 138 patients. The differences between the mutation group and the wild-type group are confirmed by t-test (Table 7).

TABLE 7
The copy number differences between the EGFR-mutant
group and wild-type group*.

| | EGF mutant | EGER wild-type | |
|---|---|---|---|
| Gene | (mean ± SD) | (mean ± SD) | p value** |
| SDK1 | 0.227 ± 0.333 | −0.040 ± 0.377 | 0.0001 |
| NFE2L3 | 0.270 ± 0.430 | −0.057 ± 0.415 | 7E−5 |
| GLI3 | 0.187 ± 0.400 | −0.140 ± 0.424 | 5E−5 |
| EGFR | 0.748 ± 1.084 | 0.399 ± 0.701 | 0.040 |
| LANCL2 | 0.562 ± 0.539 | 0.195 ± 0.363 | 3E−5 |
| VOPP1 | 0.386 ± 0.540 | 0.001 ± 0.366 | 1E−5 |

*data obtained by genomic qPCR in 114 patients.
**p value is calculated by two sample t-test.

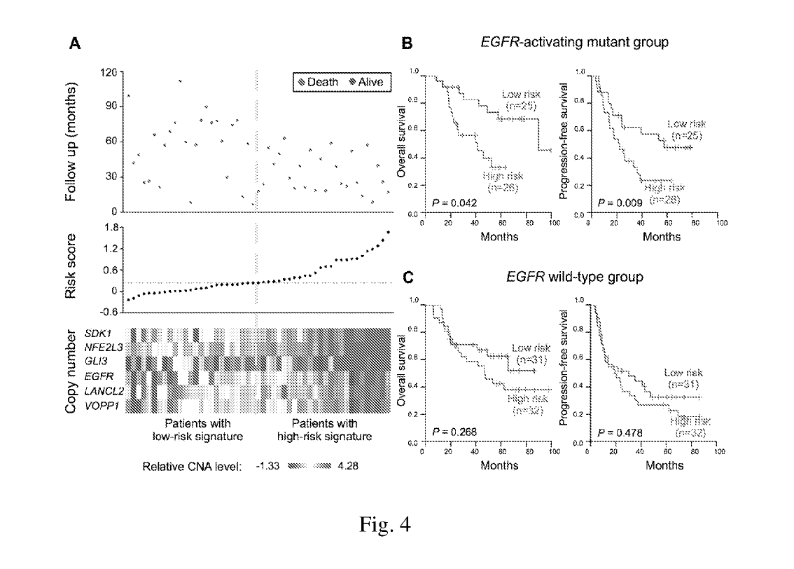

The average of the copy numbers of the six genes was used to predict the patient survival for the EGFR-mutation group (FIG. 4A). As shown in FIG. 4B, both log rank test and univariate Cox regression showed that this copy-number based risk score (CNA-risk score) is able to discriminate the high risk patients from the low-risk patients for both overall survival and progression-free survival prediction. A multivariate Cox regression was performed. The result shows that the prediction ability of our CNA-risk score is independent of cancer stage (Table 8).

TABLE 8
The multivariate Cox regression results for overall survival and
progression-free survival analyses.

| | | |
|---|---|---|
| Hazard Ratio | 95% CI | p value |

Overall survival

| | | | |
|---|---|---|---|
| CNA-risk | 4.191 | 1.611 to 10.902 | 0.003 |
| Stage | 7.901 | 2.803 to 22.276 | <0.001 |
| Age | 1.04 | 0.984 to 1.099 | 0.165 |
| Gender | 1.098 | 0.302 to 3.997 | 0.887 |
| Smoking | 2.107 | 0.725 to 6.124 | 0.171 |

Progression-free survival

| | | | |
|---|---|---|---|
| CNA-risk | 2.189 | 1.028 to 4.660 | 0.042 |
| Stage | 4.939 | 2.242 to 10.881 | <0.001 |
| Age | 1.024 | 0.981 to 1.068 | 0.279 |
| Gender | 1.732 | 0.621 to 4.832 | 0.294 |
| Smoking | 1.612 | 0.658 to 3.952 | 0.297 |

The average of the copy numbers of the same six genes was also used to predict survival for the EGFR wild-type group of patients. In sharp contrast with the results of the EGFR mutant group, both log rank test and Cox regression indicated no prediction ability at all for overall survival and progression-free survival (FIG. 4C).

The survival prediction ability for each of the six genes by univariate Cox regression was examined (Table 9).

TABLE 9
The survival prediction ability of six genes in EGFR mutation
and EGFR-wild type patients

| | | |
|---|---|---|
| Hazard | | |
| ratio | 95% CI | p value* |

EGFR mutation croup

| | | | |
|---|---|---|---|
| Overall survival | | | |
| SDK1 | 3.062 | 0.972 to 9.644 | 0.056 |
| NFE2L3 | 1.927 | 0.869 to 4.276 | 0.107 |
| GLI3 | 2.004 | 0.849 to 4.727 | 0.113 |
| EGFR | 1.943 | 1.325 to 2.849 | 0.001 |
| LANCL2 | 2.404 | 1.232 to 4.691 | 0.010 |
| VOPP2 | 2.813 | 1.429 to 5.535 | 0.003 |
| Progression-free survival | | | |
| SDK1 | 2.878 | 1.046 to 7.924 | 0.041 |
| NFE2L3 | 1.737 | 0.905 to 3.333 | 0.097 |
| GLI3 | 2.079 | 0.994 to 4.35 | 0.052 |
| EGFR | 1.302 | 0.935 to 1.814 | 0.118 |
| LANCL2 | 2.165 | 1.207 to 3.883 | 0.010 |
| VOPP2 | 2.184 | 1.237 to 3.855 | 0.007 |

Wild-type group

| | | | |
|---|---|---|---|
| Overall survival | | | |
| SDK1 | 1.491 | 0.608 to 3.658 | 0.383 |
| NFE2L3 | 1.341 | 0.588 to 3.059 | 0.486 |
| GLI3 | 1.435 | 0.645 to 3.19 | 0.376 |
| EGFR | 0.596 | 0.341 to 1.040 | 0.069 |
| LANCL2 | 1.375 | 0.537 to 3.519 | 0.507 |
| VOPP2 | 1.208 | 0.467 to 3.128 | 0.696 |
| Progression-free survival | | | |
| SDK1 | 1.055 | 0.491 to 2.270 | 0.890 |
| NFE2L3 | 1.122 | 0.567 to 2.221 | 0.741 |
| GLI3 | 1.243 | 0.661 to 2.338 | 0.499 |
| EGFR | 0.635 | 0.414 to 0.975 | 0.038 |
| LANCL2 | 1.050 | 0.491 to 2.244 | 0.900 |
| VOPP2 | 0.909 | 0.417 to 1.981 | 0.810 |

*p value is calculated by univariate cox regression.

As expected, the results showed marked differences between the EGFR mutation and EGFR wild-type groups. For the mutation group, the p-values for the six genes in overall survival and progression-free prediction are either significant (p<0.05) or marginal with the largest p-value=0.118. For the wild-type group, the p-values are much larger.

To examine the ability of the six genes in predicting a patient's drug responsiveness, two groups from 23 advanced stage lung adenocarcinoma patients with EGFR sensitive mutation (L858R or exon-19 deletion) were formed: the favorable group which consists of 11 patients with partial response (PR) and the less favorable group which consists of 12 patients with stable disease (SD) or progressive disease (PD). As shown in FIG. 4A, the average CNA of six genes is significantly smaller for the favorable response group of patients (t-test, p=0.004).

Furthermore, as shown in FIG. 4B, we found that simultaneous presence of four or more genes in this cluster with CNA higher than average is associated with less favorable drug response (n=23, Fisher exact test, p=0.0069).

What is claimed is:

1. A method for predicting the response of an EGFR-activating mutant subject suffering from a lung adenocarcinoma and receiving treatment with epidermal growth factor receptor tyrosine kinase inhibitor (EGFR-TKI), comprising a) providing a sample comprising genomic DNA from said EGFR-activating mutant subject; and b) analyzing said genomic DNA to determine copy number alterations (CNAs) of genes in chromosome 5p, 7p, 8q or 14q of the sample, wherein changes of CNAs in the sample of a) relative to a sample comprising genomic DNA of a EGFR wild-type indicate that the EGFR-activating mutant subject has less favorable response to treatment with the EGFR-TKI.

2. The method of claim 1, wherein the lung adenocarcinoma is non-small-cell lung cancer (NSCLC).

3. The method of claim 1, wherein the EGFR-TKI is gefitinib (Iressa; N-(3-Chloro-4-fluoro-phenyl)-7-methoxy-6-(3-morpholin-4-ylpropoxy)quinazo-lin-4-amine), erlotinib (Tarceva; N-(3-ethynylphenyl)-6,7-bis(2-methoxyethoxy)quinazolin-4-amine) and lapatinib (Tykerb, GW572016 or N-[3-chloro-4-[(3-fluorophenyl)methoxy]phenyl]-6-[5-[(2-methylsulfonyleth-ylamino)methyl]-2-furyl]quinazolin-4-amine).

4. The method of claim 1, wherein the EGFR-TKI is CI-1033, EKB-569 or HKI-272.

5. The method of claim 1, wherein the copy number alterations (CNAs) of genes in chromosome 7p of the sample in step b) are determined.

6. The method of claim 1, wherein the genes in step b) are in chromosome 7p11.2, 7p14.1, 7p15.2, 7p15.3, 8q11.21 or 8q11.23.

7. The method of claim 1, wherein the gene in step b) is selected from the group consisting of: EGFR, LANCL2, VSTM2A, VOPP1, SEC61G, SEPT14 and HPVC1 located at the chromosome 7p11.2, GLI3 and C7orf10 located at the chromosome 7p14.1, NFE2L3, MIR148A and OSBPL3 located at the chromosome 7p15.2, NPY located at the chromosome 7p15.3, SDK1 located at the chromosome 7p22.2, ANK1 located at the chromosome 8p11.21 and ADAM3A located at the chromosome 8p11.23.

8. The method of claim 1, wherein the gene in step b) is GLI3, NFE2L3, SDK1, EGFR, VOPP1 or LANCL2 or a combination thereof.

9. The method of claim 1, wherein the changes of CNAs are DNA gain in chromosome 5p, 7p or 14q and DNA loss in chromosome 8q.

10. A method of predicting prognosis in an EGFR-activating mutant subject suffering from a lung adenocarcinoma and receiving treatment with EGFR-TKI, comprising a) providing a sample comprising genomic DNA from said EGFR-activating mutant subject; and b) analyzing said genomic DNA to determine copy number alterations (CNAs) of genes in chromosome 5p, 7p, 8q or 14q of the sample, wherein the subject is determined to have poorer prognosis when the CNAs in the sample of a) is changed relative to the CNAs of genes in a sample comprising genomic DNA of a EGFR wild-type.

11. The method of claim 10, wherein the lung adenocarcinoma is non-small-cell lung cancer (NSCLC).

12. The method of claim 10, wherein the EGFR-TKI is gefitinib (Iressa; N-(3-Chloro-4-fluoro-phenyl)-7-methoxy-6-(3-morpholin-4-ylpropoxy)quinazo-lin-4-amine), erlotinib (Tarceva; N-(3-ethynylphenyl)-6,7-bis(2-methoxyethoxy)quinazolin-4-amine) and lapatinib (Tykerb, GW572016 or N-[3-chloro-4-[(3-fluorophenyl)methoxy]phenyl]-6-[5-[(2-methylsulfonyleth-ylamino)methyl]-2-furyl]quinazolin-4-amine).

13. The method of claim 10, wherein the EGFR-TKI is CI-1033, EKB-569 or HKI-272.

14. The method of claim 10, wherein the copy number alterations (CNAs) of genes in chromosome 7p of the sample in step b) are determined.

15. The method of claim 10, wherein the genes in step b) are in chromosome 7p11.2, 7p14.1, 7p15.2, 7p15.3, 8q11.21 or 8q11.23.

16. The method of claim 10, wherein the gene in step b) is selected from the group consisting of: EGFR, LANCL2, VSTM2A, VOPP1, SEC61G, SEPT14 and HPVC1 located at the chromosome 7p11.2, GLI3 and C7orf10 located at the chromosome 7p14.1, NFE2L3, MIR148A and OSBPL3 located at the chromosome 7p15.2, NPY located at the chromosome 7p15.3, SDK1 located at the chromosome 7p22.2, ANK1 located at the chromosome 8p11.21 and ADAM3A located at the chromosome 8p11.23.

17. The method of claim 10, wherein the gene in step b) is GLI3, NFE2L3, SDK1, EGFR, VOPP1 or LANCL2 or a combination thereof.

18. The method of claim 10, wherein the changes of CNAs are DNA gain in chromosome 5p, 7p or 14q and DNA loss in chromosome 8q.

19. A diagnostic kit for determining the response of an EGFR-activating mutant subject suffering from lung adenocarcinoma and receiving treatment with EGFR-TKI, or determining prognosis in a EGFR-activating mutant subject suffering from a lung adenocarcinoma and receiving treatment with EGFR-TKI, comprising one or more probes to the genes in chromosome 5p, 7p, 8q or 14q of the sample comprising genomic DNA from said EGFR-activating mutant subject.

20. The diagnostic kit of claim 19, wherein the lung adenocarcinoma is non-small-cell lung cancer (NSCLC).

21. The diagnostic kit of claim 19, wherein the EGFR-TKI is gefitinib (Iressa; N-(3-Chloro-4-fluoro-phenyl)-7-methoxy-6-(3-morpholin-4-ylpropoxy)quinazo-lin-4-amine), erlotinib (Tarceva; N-(3-ethynylphenyl)-6,7-bis(2-methoxyethoxy)quinazolin-4-amine) and lapatinib (Tykerb, GW572016 or N-[3-chloro-4-[(3-fluorophenyl)methoxy]phenyl]-6-[5-[(2-methylsulfonyleth-ylamino)methyl]-2-furyl]quinazolin-4-amine).

22. The diagnostic kit of claim 19, wherein the EGFR-TKI is CI-1033, EKB-569 or HKI-272.

23. The diagnostic kit of claim 19, wherein the genes are in chromosome 7p.

24. The diagnostic kit of claim 19, wherein the genes are in chromosome 7p11.2, 7p14.1, 7p15.2, 7p15.3, 8q11.21 or 8q11.23.

25. The diagnostic kit of claim 19, wherein the gene in step b) is selected from the group consisting of: EGFR, LANCL2, VSTM2A, VOPP1, SEC61G, SEPT14 and HPVC1 located at the chromosome 7p11.2, GLI3 and C7orf10 located at the chromosome 7p14.1, NFE2L3, MIR148A and OSBPL3 located at the chromosome 7p15.2, NPY located at the chromosome 7p15.3, SDK1 located at the chromosome 7p22.2, ANK1 located at the chromosome 8p11.21 and ADAM3A located at the chromosome 8p11.23.

26. The diagnostic kit of claim 19, wherein the genes in chromosome 7p is GLI3, NFE2L3, SDK1, EGFR, VOPP1 or LANCL2 or a combination thereof.

27. The diagnostic kit of claim 19, wherein the changes of CNAs are DNA gain in chromosome 5p, 7p or 14q and DNA loss in chromosome 8q.
